(19) United States
(12) Patent Application Publication
(10) Pub. No.: US 2016/0361196 A1
(43) Pub. Date: Dec. 15, 2016

(54) ATHLETIC COOLING AND HEATING SYSTEMS, DEVICES AND METHODS

(71) Applicant: Aquilo Sports LLC, Louisville, KY (US)

(72) Inventors: Paul A. Spence, Louisville, KY (US); Landon H. Tompkins, Louisville, KY (US); Samuel C. Walling, Louisville, KY (US); Ryan D. Hatton, Louisville, KY (US); Jens Hutzenlaub, Aachen (DE); William Spence, Louisville, KY (US); Alex Singer, Crestwood, KY (US)

(21) Appl. No.: 15/248,674

(22) Filed: Aug. 26, 2016

Related U.S. Application Data
(62) Division of application No. 14/127,054, filed on Apr. 9, 2014.
(60) Provisional application No. 61/572,696, filed on Jul. 20, 2011.

(51) Int. Cl. A61F 7/02; A61F 7/08; A41D 13/005; A61F 7/00

(52) U.S. Cl. CPC A61F 7/02; A61F 7/007; A61F 7/08; A61F 7/0085; A41D 13/0053; A61F 2007/0018; A61F 2007/0031; A61F 2007/0039; A61F 2007/0054; A61F 2007/0056; A61F 2007/0095; A61F 2007/0234; A61F 2007/0236; A61F 2007/0273

(57) ABSTRACT

Thermal pads for incorporation into systems and compression garments may be used for various medical or other purposes. The pads include thermal fluid channels and the ability to flex and elastically stretch in multiple directions for conformance to the individual user. Cooling systems include recirculation of the thermal fluid for temperature control purposes. Methods include hot and cold contrast type therapy for a variety of purposes.

### CROSS-REFERENCE TO RELATED APPLICATION

This application is a divisional of application Ser. No. 14/127,054 filed Apr. 9, 2014 (pending) which is a U.S. National Stage under 35 U.S.C. §371 of PCT/US2012/047428, filed Jul. 19, 2012 (expired), which claims the priority of U.S. Provisional Patent Application Ser. No. 61/572,696, filed on Jul. 20, 2011 (expired), the disclosures of which are incorporated by reference herein.

### BACKGROUND

It is well known that cooling of an injury site (usually with ice) after injury is important in reducing the swelling and inflammation around the site of injury and speeding recovery. After the acute phase has passed, recovery is often aided by episodes of heating.

In recent years, athletes have also learned about the importance of using cooling to assist in recovery after training. Cooling reduces pain, reduces tissue damage and has also been shown to have a positive effect on performance. Many high level athletes routinely use ice baths after all training sessions and games. These can take the form of large tubs filled with cold water often supplemented with ice. Other systems are available that feature tubs of water that are actively cooled. Athletes are encouraged to submerge themselves in the water to the point that muscles which were vigorously exercised are covered. Usually the athletes are asked to stay under the water for 10 to 15 minutes. The water temperature varies widely but generally ranges from several degrees above freezing to about 17° C.

Hygiene is a very important problem with ice baths. Despite chemical treatments with disinfectants and despite frequent replacement of the water in the tub, the water baths can harbor dangerous pathogens. There are reports of Staph infections (and others) that have been spread from athlete to athlete who shared a bath. Many of these athletes have small and often unrecognized breaks in their skin surface. While sitting in the water bath, pathogens can enter into the subcutaneous tissues and cause serious, and sometimes life-threatening infections that spread in limbs and even throughout the athlete's body.

So despite the fact that athletes and trainers do recognize the importance of cooling, the pain, discomfort and the dislike for sharing water between multiple users reduces compliance with this most effective therapy.

The actual medical benefits of cooling have also been assessed. Myoglobin is the principal muscle protein and creatine kinase is an important enzyme within muscles. Both myoglobin and creatine kinase are released into the blood stream after exercise and their levels remain elevated for up to 48 hours after vigorous activity. The athletes who use cooling after exercise have reduced levels of myoglobin and creatine kinase compared to those who do not. It appears that cooling reduces muscle damage and this may explain, at least in part, why cooling reduces pain and improves performance.

Temperature control of ice baths is also difficult. The temperature of the bath often varies depending on the room temperature and it frequently rises with each user. Even with active cooling of the water by refrigeration systems, it is difficult to precisely control the water temperature. It is likely that each athlete may have a slight difference in optimal cooling temperature and group-cooling baths cannot provide consistent or customizable temperature levels.

Athletes have also discovered the importance of alternating cooling and heating therapies—so called contrast therapy. Baseball pitchers are known to switch between cooling and heating tanks to submerge their shoulder and arm region for segmented periods of several minutes each. It would be useful to have an improved system for providing contrast therapy.

Ice tubs and cooling baths make it difficult to target therapy to specific areas. While it is reasonably easy for an athlete to cool his or her legs, cooling the back of a pole vaulter or weight lifter, or the shoulder and back area of a pitcher is not easy without submerging the entire body in the tub. Providing better manners to cool in precise locations would also be an advantage.

Portability can be a problem with the current standard of cooling therapy. Ice tubs and ice baths in locker rooms cannot be moved easily. Since many professional teams and elite athletes are frequently away from their home training site it would be very useful if they could cool anywhere.

Various cooling garments and wraps, such as those disclosed in U.S. Pat. No. 7,107,629 and U.S. Patent Publication No. 2011/0098793 are known. However, even with these devices that address some of the issues of ice water baths, improvements would be desirable.

### SUMMARY

The invention has many various aspects, and this summary is provided for a few of the aspects, while additional aspects will be apparent from a review of the illustrative examples provided in the more detailed description to follow, as well as the drawings.

In one illustrative embodiment, a pad is provided for heating and/or cooling one or more body parts of an individual. The pad includes a first flexible layer of material, and a second flexible layer of material secured to the first flexible layer of material. These layers may be formed of any suitable material, and exemplary materials will be polymeric or plastic, waterproof materials. A plurality of spaced thermal fluid passages is formed between the first and second flexible layers of material. An inlet is in fluid communication with the plurality of spaced thermal fluid passages, and an outlet is in fluid communication with the plurality of spaced thermal fluid passages. Thermal fluid, such as water or another liquid, may be directed from the inlet into the plurality of thermal fluid passages and exit the outlet after travelling through the pad. A plurality of elongate openings are in the pad, and at least some of the elongate openings are positioned in spaces between the thermal fluid passages. The elongate openings allow the pad to be flexed and elastically stretched in multiple directions to accommodate the one or more body parts of the individual.

The pads of this invention may be formed in many different ways and may have many other features as contemplated herein, and these features may be added to or substituted for those described immediately above. As some examples, the pad may have an elongate opening dividing the pad into first and second sections for allowing the pad to be wrapped around a body part. The pad may have a perimeter with discontinuities for providing flexibility at the perimeter of the pad. The fluid passages may further comprise channels that include at least two sections separated by a flexible membrane. One section contains the thermal fluid passages and the other section is adapted to receive a compression fluid, such as pressurized air for purposes to be described below.

In another illustrative embodiment, the invention provides a system for cooling one or more body parts of an individual. Generally, the system includes a cooling unit for holding and cooling a thermal fluid, a garment configured to receive the one or more body parts, and at least one cooling pad associated with the garment. The cooling pad includes a plurality of spaced thermal fluid passages, an inlet in fluid communication with the plurality of spaced thermal fluid passages, and an outlet in fluid communication with the plurality of spaced thermal fluid passages. Fluid may be directed from the inlet into the plurality of thermal fluid passages and exit the outlet after travelling through the pad. The system also includes a supply conduit coupled with the inlet, a return conduit coupled with the outlet, and a pump fluidly coupled with the cooling unit to drive the thermal fluid in a fluid path from the cooling unit, through the inlet, supply conduit, thermal fluid passages, outlet, and return conduit, thereby circulating the cooled thermal fluid within the cooling pad. In accordance with one aspect, a recirculation passage is fluidly coupled between the inlet and the outlet for directing an amount of the thermal fluid that has been circulated in the pad back into the inlet in order to raise the temperature of the cooled thermal fluid to a temperature above the temperature produced by the cooling unit. As will be appreciated from a further review of the description to follow, this allows the temperature to be raised to a slightly higher level to provide adequate cooling to the individual while maintaining better comfort.

Various other systems including aspects discussed immediately above in addition to other aspects for facilitating other functions and results are also discussed herein. Relative to the recirculation feature, additional aspects may include the use of a flow control device coupled in the recirculation passage for controlling the amount of thermal fluid directed back into the inlet. The flow control device may further comprise an adjustable flow control device that is capable of selectively adjusting the amount of thermal fluid directed back into the inlet. Optionally, the flow control device may further comprise a non-adjustable flow control device, such as a shunt, that directs a predetermined amount of the thermal fluid back into the inlet.

The present invention also provides a variety of methods for therapeutic or other purposes. Various methods will become apparent from a review of the detailed description and drawings. An illustrative embodiment for providing hot and cold therapy to one or more body parts of an individual utilizes a pad as generally described. The method includes delivering cold fluid to the spaced thermal fluid passages of the pad while the pad is applied to one or more body parts, and applying heat to one or more body parts with the pad. Applying heat may further comprise delivering hot fluid to the spaced thermal fluid passages of the pad while the pad is applied to one or more body parts. Optionally, applying heat may include doing so from an electric heater affixed to the pad while the pad is applied to one or more body parts.

Various additional advantages and features will become more readily apparent to those of ordinary skill in the art upon review of the following detailed description of the illustrative embodiments taken in conjunction with the accompanying drawings.

### BRIEF DESCRIPTION OF THE DRAWINGS

FIG. 1 is an illustration of a first illustrative embodiment of a system constructed in accordance with the invention coupled to an individual.

FIG. 2A is a schematic illustration of another embodiment of a system applied to an individual's arm.

FIG. 2B is a schematic illustration of another system with a cooling pad/garment applied to the torso of an individual.

FIG. 2C is a fragmented and cross sectioned view of the garment and pad shown in FIG. 2A.

FIG. 2D is a cross sectional, fragmented view similar to FIG. 2C, but showing another alternative.

FIG. 2E is a schematic illustration similar to FIGS. 2C and 2D, but illustrating another embodiment.

FIG. 3A is a perspective view of a compression garment, including an inner liner, an outer shell and a pair of pads for cooling and/or heating purposes.

FIG. 3B is an assembled view of the garment illustrated in FIG. 3A.

FIG. 4 is a cross sectional view taken along line 4-4 of FIG. 3B.

FIG. 5 is a cross sectional view of another embodiment of a pad/garment structure.

FIG. 6A is a cross sectional view of another pad/garment structure.

FIG. 6B is a cross sectional view of the pad/garment structure shown in FIG. 6A, but in a stretched or expanded condition.

FIGS. 7A and 7B are fragmented, cross sectional views of an embodiment in which an elastic portion connects the pads in a garment structure.

FIGS. 8A and 8B illustrate two different embodiments for pads constructed in accordance with the invention.

FIG. 9 is a schematic, partially fragmented view of a compression garment constructed in accordance with an illustrative embodiment, and including various pads for heating and/or cooling purposes.

FIG. 10A is a view similar to FIG. 9, but illustrating another embodiment of a cooling and/or heating pad structure.

FIG. 10B is an elevational view of the cooling and/or heating pad shown in FIG. 10A.

FIG. 10C is an enlarged view of the pad shown in FIGS. 10A and 10B illustrating additional detail and thermal fluid flow paths.

FIG. 10D is a cross sectional view taken along line 10D-10D of FIG. 10C.

FIG. 10E is a cross sectional view similar to FIG. 10D, but illustrating the pad in a stretched condition.

FIG. 11A is an elevational front view of a compression garment for the upper body of an individual, including a cooling and/or heating pad structure designed to fit over the arm and right shoulder/back region of the individual.

FIG. 11B is an elevational rear view of the compression garment shown in FIG. 11A.

FIG. 12 is a schematic illustration of a first illustrative embodiment of a system including a recirculation path for temperature control of the cold fluid.

FIG. 13 is a schematic view of an alternative system similar to the system shown in FIG. 12.

FIG. 14 is a schematic illustration illustrating another embodiment of a system for recirculating fluid and temperature control.

FIG. 15 is a schematic illustration illustrating another system for recirculating fluid and temperature control.

FIG. 16A is a perspective view of another alternative compression garment structure, illustrating the inner liner and a pair of pad structures that include both cooling and heating.

FIG. 16B is another embodiment of a compression garment structure including additional alternatives for heating and cooling therapy to the individual.

FIG. 17 is a perspective view of another alternative garment including both heating and cooling capabilities.

FIGS. 18A, 18B and 18C are respective alternative embodiments for hot and cold fluid reservoirs usable with the systems described.

FIGS. 19A, 19B and 19C illustrate, schematically, different set-ups for use of hot and cold fluid reservoirs during therapies or sessions in which the body is subjected to hot and cold compression therapy.

FIGS. 20A, 20B and 20C schematically illustrate another alternative system for providing contrast or hot/cold therapy.

FIG. 20D is a schematic illustration of another alternative system similar to that shown in FIGS. 20A-20C.

FIG. 21 is a schematic illustration of another alternative system similar to that shown in FIGS. 20A-20C.

FIGS. 21A, 21B and 21C are cross sectional illustrations of the pad/garment structure usable with the system shown in FIG. 21.

FIGS. 22A, 22B and 22C are schematic illustrations of another alternative system for providing contrast or hot/cold therapy to an individual.

FIGS. 23A, 23B and 23C are respective cross sectional views of a pad/garment structure in alternative forms for providing hot and/or cold therapy to the individual.

FIG. 24 is a schematic illustration of another illustrative system for providing hot and/or cold therapy to an individual.

FIGS. 25A, 25B, 25C and 25D are schematic illustrations of another system constructed for providing hot and/or cold therapy to an individual.

FIG. 26 is an elevational view of another alternative pad structure for providing hot and/or cold therapy to an individual.

FIG. 26A is a cross sectional view taken along line 26A-26A of FIG. 26.

FIG. 26B is a cross sectional view of the pad shown in FIG. 26A, but illustrating the pad in a stretched condition.

FIGS. 27 through 34 are respective elevational views of additional, alternative embodiments of cooling and/or heating pads constructed in accordance with the invention.

FIG. 35 is an elevational view of another alternative pad for hot and/or cold therapy.

FIG. 35A is an enlarged view of the section marked “35A” in FIG. 35.

FIG. 36 is a schematic illustration of another system for providing cold therapy, and using a recirculation path for temperature control purposes.

FIG. 37 is a schematic illustration of another system for providing cold therapy, and using a recirculation path for temperature control purposes.

FIG. 38 is a schematic illustration of another system for providing cold therapy, and using a recirculation path for temperature control purposes.

FIGS. 39A, 39B and 39C is a schematic illustration, partially in cross section illustrating another alternative recirculation path and flow control device, with the flow control device in different positions in each figure.

FIG. 40A is a schematic illustration similar to FIGS. 39A-C, but illustrating an alternative in which the recirculation path is formed by a coiled conduit.

FIG. 40B is an illustration similar to FIG. 40A, but showing an alternative embodiment.

FIG. 40C is a schematic illustration similar to FIG. 40B, but illustrating another alternative embodiment.

FIG. 41 is a schematic, cross sectional view of an integrated cold fluid reservoir, fluid components and control unit usable with the present invention.

FIGS. 42 through 44 are respective elevational views of additional, alternative embodiments of cooling and/or heating pads constructed in accordance with the invention.

### DETAILED DESCRIPTION

Throughout the detailed description, like reference numerals refer to like elements of structure and function. Therefore, for brevity, such elements may not be described more than once.

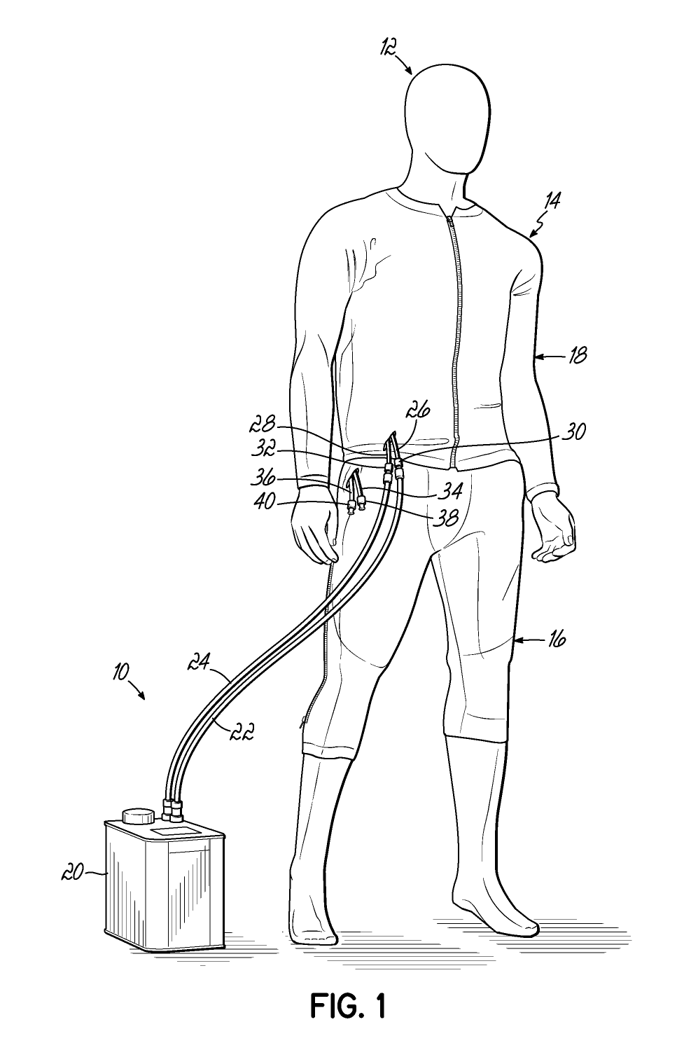

FIG. 1 illustrates a general system 10 constructed in accordance with an embodiment of the invention. This system 10 may have any one or more of the features to be described hereinbelow. This is a particularly useful way of providing cyclotherapy and/or heat therapy for any individual 12, such as an athlete or patient. The individual 12 is shown with a compression suit 14 including pants 16 and a jacket or shirt 18. Cooling/heating pads, as further described herein, are attached or otherwise used with or integrated with the compression suit 14. The compression may be provided by the material of the suit 14 itself such as by the use of any suitable stretch material, or may be provided in a more active manner through the use of, for example, pneumatic or hydraulic compression systems and methods. The system 10 includes a cooling unit 20, which may be a highly controllable electronic unit that utilizes thermoelectric cooling devices, or may be a more economically practical cooling unit that uses a thermally insulated cooler containing, for example, ice water. As will be appreciated from the description to follow, the cooling unit 20 may optionally include heating capability as well or electric heaters may be incorporated for various purposes and therapies. It will be appreciated that the power supply or supplies for running the various electrical components may be portable or not depending on need. The cooling units associated with this disclosure may have multiple sources of cooling, such as ice water, Peltier devices and refrigeration units. The units may be designed to service multiple users for group therapy. The cooling unit 20 is coupled to the suit 14 to supply cooled fluid, such as water, through a flexible inlet or input conduit 22 and is continuously cycled or pumped through the suit 14 and out from an outlet or output conduit 24. This illustrative suit 14 includes respective inlet and outlet conduits 26, 28 and fluid couplings 30, 32 enabling the inlet and outlet conduits 22, 24 from the cooling unit 20 to be suitably coupled to the suit 14. In this example, respective inlet and outlet conduits 26, 28 and couplings 30, 32 are provided for the upper piece of apparel, i.e., the shirt or jacket 18, and similar inlet and outlet conduits 34, 36 and couplings 38, 40 are provided for the lower piece of apparel, i.e., the pants 16. The conduits may be flexible silicone tubes. As will be further described below, the cooling unit 20 may have associated controls and may contain, in a single location, the cooling fluid such as water, the cooling mechanism, such as thermoelectric cooling devices, a refrigeration unit, or simply ice, as well as other electronic and fluid control components such as one or more flow control devices or valves, one or more static shunts, and the necessary pump or pumps for driving the fluid along the fluid circuit, including into and out of the cooling pads (not shown) associated with the suit components.

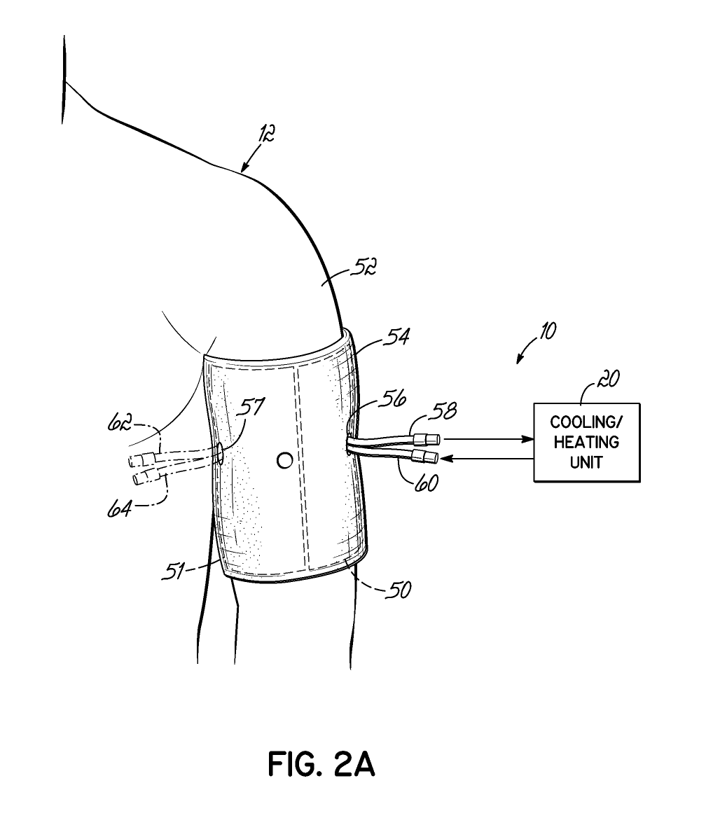

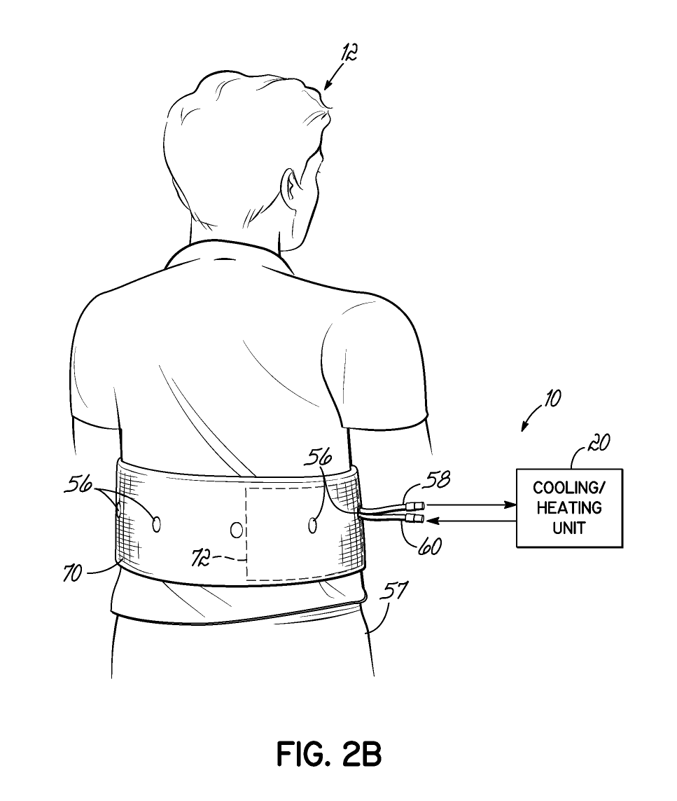

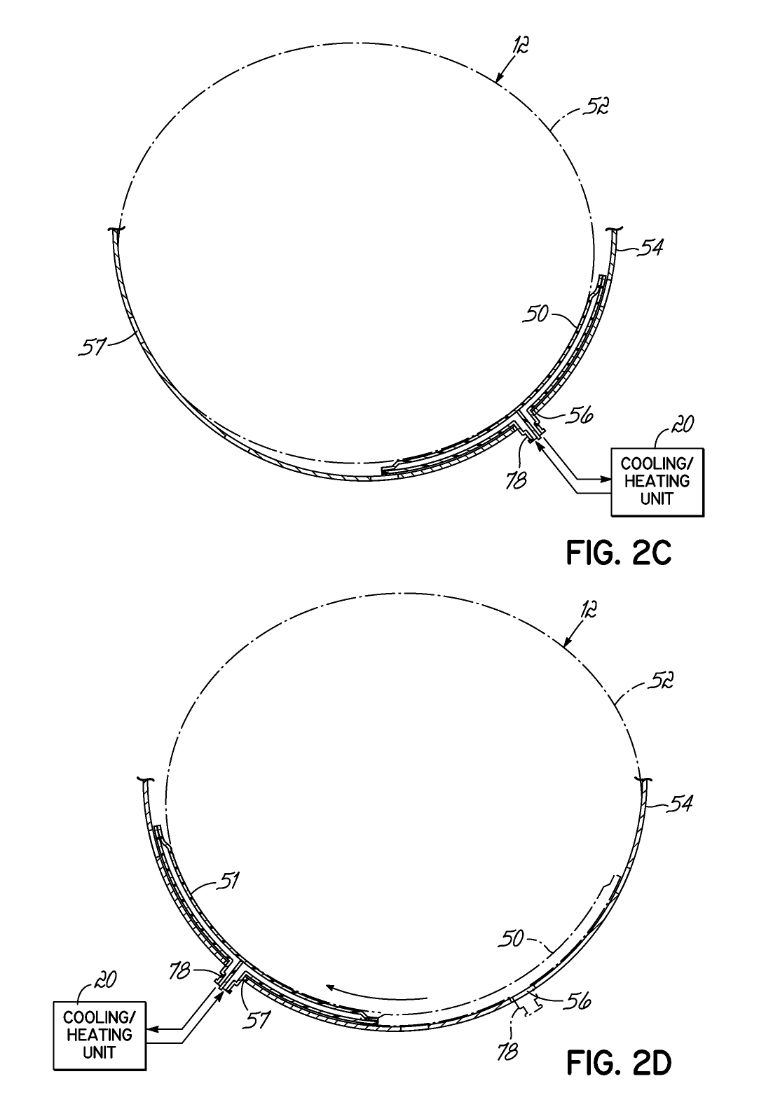

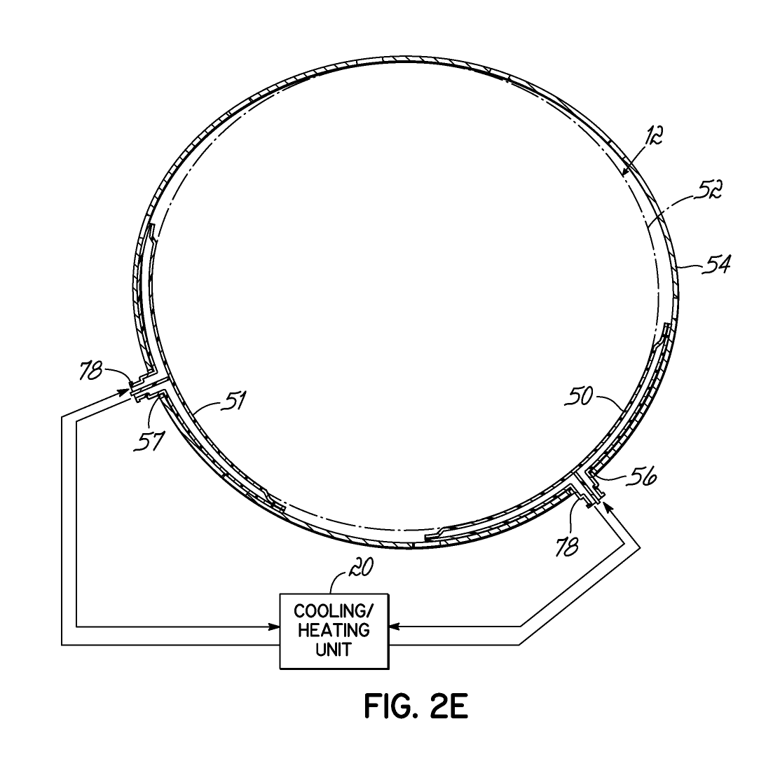

FIG. 2A illustrates another embodiment of the invention in which a very localized portion of the individual 12 is subjected to cooling and/or heating from a system 10 as described herein. In this example, cooling pads 50, 51 surround an upper arm 52 of the individual 12. Here, the cooling pads 50, 52 is held by a sleeve 54 and the sleeve 54 may be made from a material that provides compression, such as polyester and LYCRA® brand fiber or a combination of these or other materials. The sleeve 54 may include apertures and passages 56, 57 to accommodate the tubes or conduits 58, 60 for the heating and/or cooling fluids directed to the pad 50. Additional input and output conduits 62, 64 may be provided for coupling to the pad 51 to allow easier hot/cold therapy by the respective pads 50, 51. The pads 50, 51 may be constructed such that the tubes enter and exit the sleeve 54 at right angles to the pad in order to help prevent kinking. The compression sleeve 54 and pad combination 50, 51 may be formed as separate components or formed integrally, and may include any of various ways to attach the sleeve/pad to the individual, including zippers or other fasteners, such as hook-and-loop type fasteners. The sleeve 54 may be formed as a cylindrical compression piece through which the individual's arm 52 or other limb is inserted. Other manners of increasing compression may be used such as separate elastic bands, snaps, magnets or pneumatic/hydraulic systems. As will be appreciated from the description to follow, if the pads 50, 51 that hold the fluid are formed as separate components from the apparel portion of the sleeve 54 or other item, they may be held in place in various manners, such as through the use of snap fasteners, hook-and-loop material, or magnets. The cooling pads 50, 51 themselves, shown in dashed lines, may be of any desired shape and size and may even extend beyond the margin of the sleeve 54 or other item of apparel itself. For purposes of fitting well to other portions of the body, such as joints, the sleeve 54 may be preformed with a suitable bend to accommodate the desired area of the body, such as an ankle joint or knee joint. Cooling and heating may be used in a cyclic fashion to provide contrast therapy or other uses as further described below. For this purpose, the cooling unit 20 described with regard to FIG. 1 may in various embodiments comprise a cooling and heating unit that allows cooled fluid to be cycled into the compression suit or other more localized item of apparel, and then purged or removed from the apparel, followed by cycling of heating fluid or vice versa. As another option, one portion of a individual's body may be cooled while another portion of the individual's body may be heated.

FIG. 2B is similar to FIG. 2A, but illustrates another alternative for localized cooling and/or heating of an individual's body. Here, a sleeve 70 is constructed for fitting around an individual's waist and/or abdominal and back region and includes at least one heating and/or cooling pad 72 (e.g., a thermal pad for receiving heated or cooled fluid). This sleeve 70 may be moved vertically to apply cooling and/or heating to different locations along the individual's torso.

FIG. 2C is a schematic cross sectional view of the item of apparel or sleeve 54 shown in FIG. 2A. Here, a single cooling pad 50 is shown and compressed against the individual 12 (dash-dot lines). The pad is held against the individual's body by the compressive material of the sleeve 54, and a fluid coupling 78 exits at least substantially perpendicular to the pad 50. It will be appreciated, however, that the fluid couplings for the pad or pads may be located at any desirable position and orientation relative to the associated pad.

FIG. 2D is another cross sectional view, similar to FIG. 2C, but illustrating that the fluid coupling 78 associated with the pad 50 may be changed to different points along the sleeve 54, or that multiple fluid couplings 78 may be incorporated into the pad or pads 50, 51. For example, this allows different shaped pads 50, 51 to be used with a sleeve 54 that fits over a particular region. The pads 50, 51 and elastic compression sleeve 54 may be integrated or attached to one another permanently such as by sewing or other adhesive or welding methods, or may be detachable from one another for easier cleaning and replacement or repair. In addition, although only a single layer or compression fabric is shown, with the pads 50, 51 on the inside surface of the elastic or compression fabric sleeve 54, it will be understood that an additional layer of compression or other fabric (not shown) may be used on the inside of the pads 50, 51 such that the pads 50, 51 are sandwiched between two layers of fabric. This may provide a more comfortable layer against the skin of the individual. The inner layer or liner may be soft for comfort against the skin.

FIG. 2E illustrates a cross section of the heating and cooling devices similar to FIG. 2D. Here, a single compression sleeve 54 of fabric holds separate cooling and heating pads 50, 51.

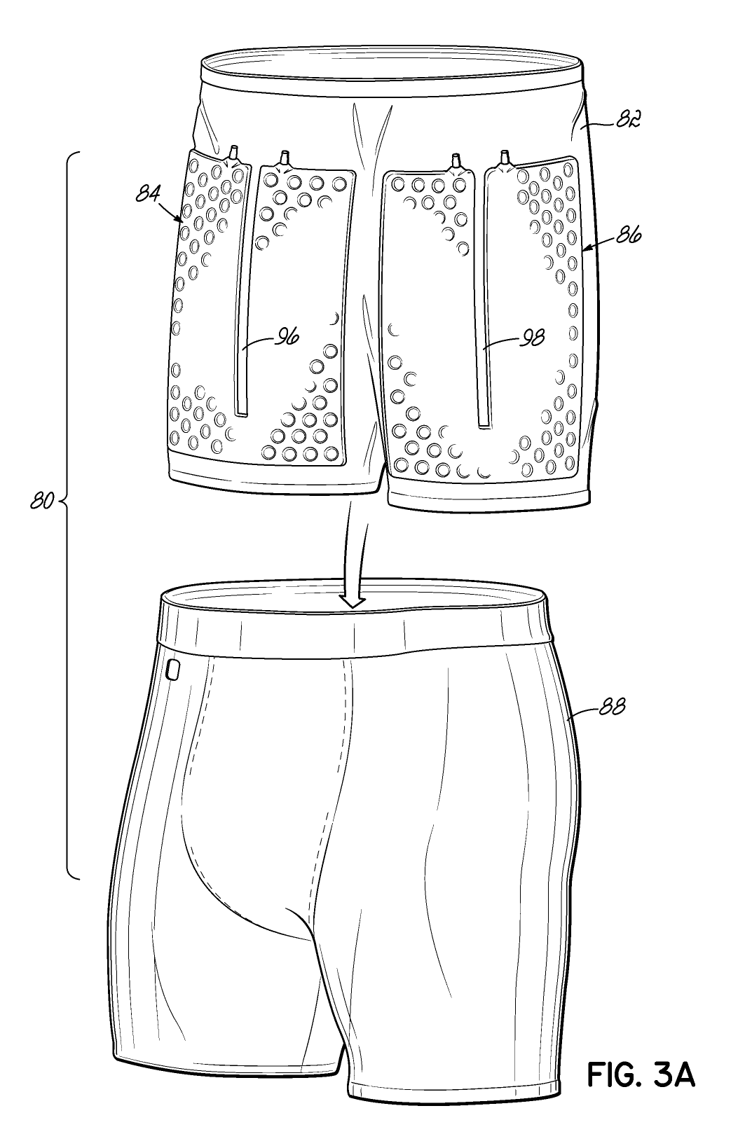

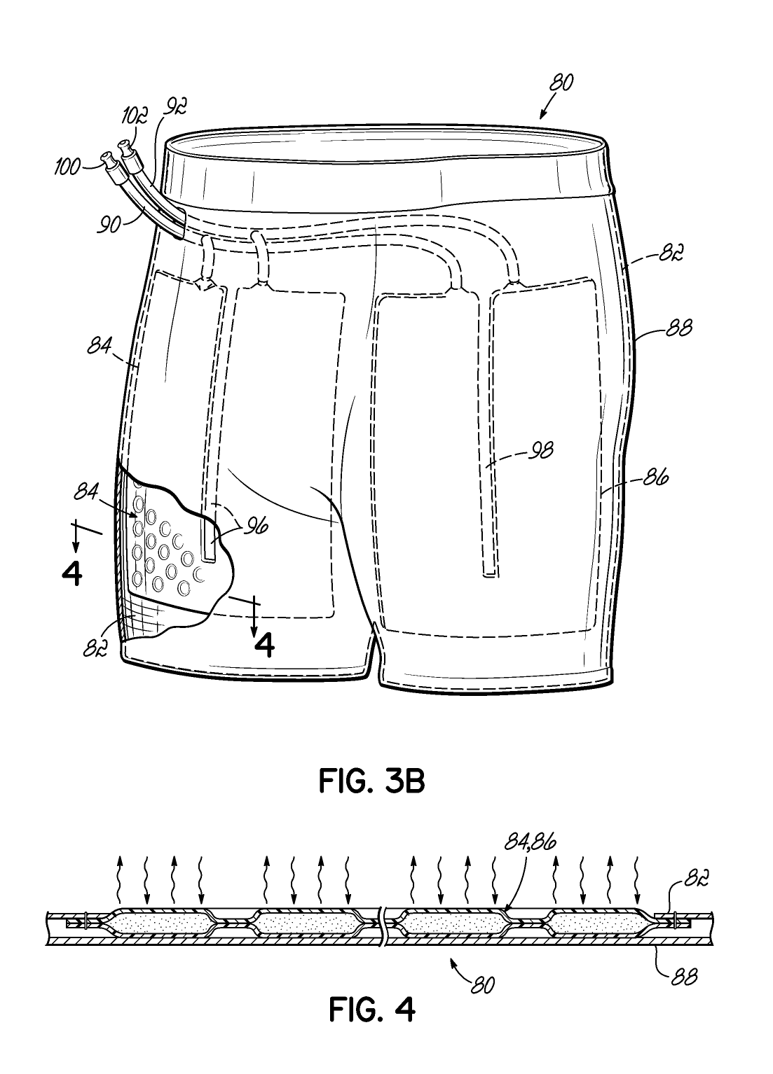

FIG. 3A illustrates the formation of a comfortable cooling and/or heating garment 80. In this example, a two-layer garment 80 is shown, with the inner layer or liner 82 supporting multiple thermal pads 84, 86. As previously mentioned, the pads 84, 86 may be affixed to the inner liner 82 of suitable fabric, which may or may not be elastic stretch material to provide compression. The pads 84, 86 may be attached to the inner liner 82 in any suitable manner, such as by sewing, adhesive, or fasteners such as snaps, hook-and-loop material or magnets. The pads 84, 86 may contact the skin of the individual directly, such as by providing apertures in the inner liner 82, or the pads 84, 86 may provide the heating and cooling effect via the inner liner 82 itself. In this manner, the inner liner 82 may provide a varying degree of insulation for the individual in order to prevent too much cooling or heating directly against the skin. The inner liner 82 is slipped inside of an outer garment layer or shell 88, and the two layers 82, 88 may be held together by compression or may be more directly affixed to one another by sewing, or affixed in any other permanent or temporary manner, such as through the use of suitable fasteners. The tubes (not shown) that are necessary for transmitting the heating and/or cooling fluid may be positioned between the two layers 82, 88 and, therefore, a more refined appearance to the garment 80 is provided. The pads 84, 86 may be designed, configured and placed to provide cooling and/or heating, as well as avoid cooling and/or heating to any desired region of the individual's body. The inner and outer garments or layers 82, 88 may be generally the same shape, as shown, or of different shapes. For example, the inner liner 82 of this example may not need a waistband as shown. The level of compression may be altered by selecting the size and level of compression force of the material used to create the inner liner 82 and the outer garment or shell 88.

FIG. 3B illustrates the fully assembled garment 80 of FIG. 3A. Fluid tubes 90, 92 exit from a small opening 94 in the outer shell 88 for inflow and outflow of circulated cooling and/or heating fluid. In this embodiment, the pads may be obtained, for example, from Cincinnati Sub-Zero, Cincinnati, Ohio. However, the pads have been improved with one or more slits 96, 98 to provide expansion and flexibility to accommodate individuals of different shapes and sizes. The flow pattern for the two pads 84, 86 shown in the drawing, which cover the front thighs of the individual, flows into one side of each pad 84, 86 from the inlet conduit 90, travels to the bottom of that pad 84, 86, and then returns up the other side of the pad 84 or 86 into the fluid exit or outlet conduit 92. Additional external conduits (not shown) are respectively coupled to the fluid couplings 100, 102 outside of the garment 80, preferably, and are coupled to the cooling and/or heating unit 20 (FIG. 1). It will be understood that additional pads may be added to the other sides of the thighs or any other area covered by the garment 80, or the pads 84, 86 as shown in FIG. 3B may be extended in size to cover additional desired areas of the body. In another alternative, a single integrated or separate cooling pad may be used to cover the entire desired area of the individual, e.g., a single pad that envelops or surrounds a single leg or, individually, both legs. The various internal tubes or conduits associated with each pad are preferably fluidly coupled together such that only a single pair of conduits 90, 92 extend from the garment 80, as shown, as opposed to having multiple conduits exit the garment 80.

FIG. 4 is a cross sectional view of FIG. 3B, as shown. This figure shows two complete layers 82, 88 of the garment 80, which sandwich the pads 84, 86. Apertures in the inner layer 82 allow the cooling and/or heating pads 84, 86 to make direct contact with the individual wearing the garment 80. This may provide better heat transfer in those situations as necessary.

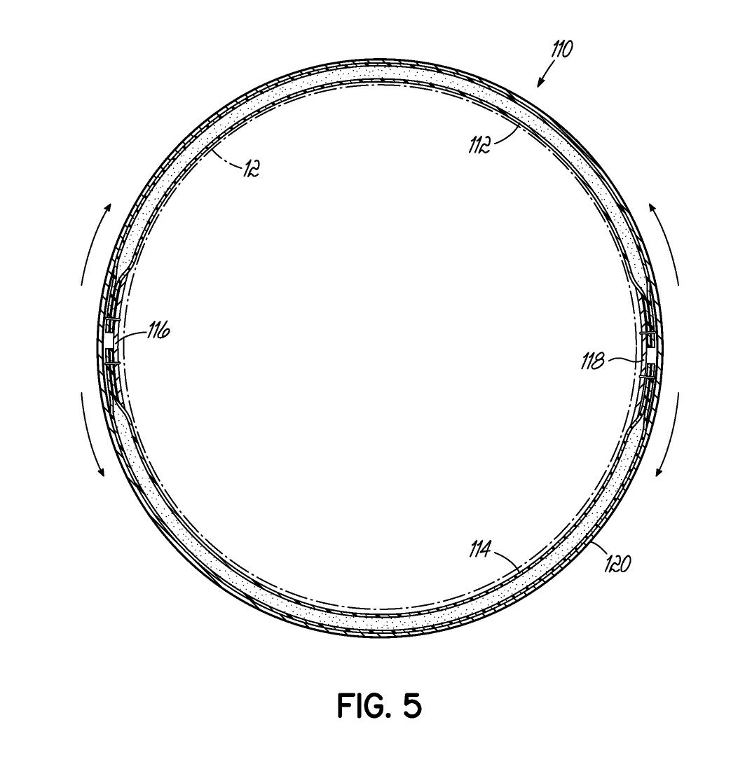

FIG. 5 is a cross sectional view of a cylindrical garment 110, similar to the garment illustrated in FIG. 2A, but showing thermal pads 112, 114 on the front and back sides or opposite sides of the garment 110. Unless the pad is modified, such as in one of the manners describe below, the pad typically will have limited or no flexibility or elasticity. To conform to different individuals and to maintain good compression, various options may be used. In FIG. 5, relatively rigid front and rear cooling and/or heating pads 112, 114 are shown. The pads 112, 114 are connected by elastic material portions 116, 118 which allow the pads 112, 114 to expand apart. The pads 112, 114 are covered on the outside surfaces with a compression (elastic) shell 120, such as a shell as previously described, to maintain the pads 112, 114 in compressive contact with the individual 12. In this example, no inner liner is shown, and the pads 112, 114 directly contact the individual 12. Optionally, an inner liner may be added to this arrangement and may or may not be formed from compressive or elastic material. If a nonelastic material is used, the inner liner may simply serve as a soft material for comfort purposes against the skin of the individual user. Additional compression may be provided by having both inner and outer layers formed from a compressive, elastic material such as SPANDEX® brand fabric or other material.

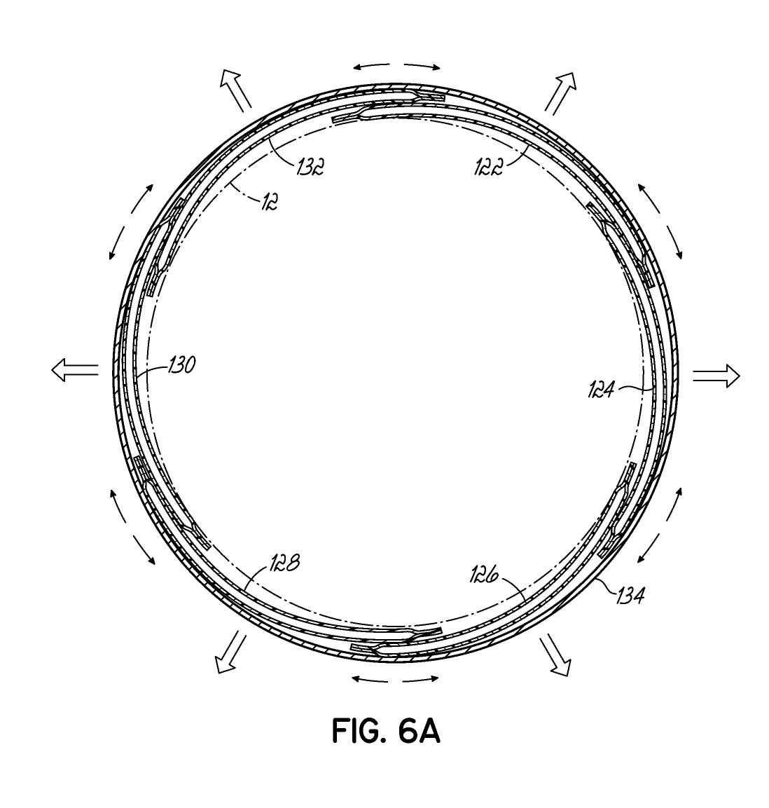

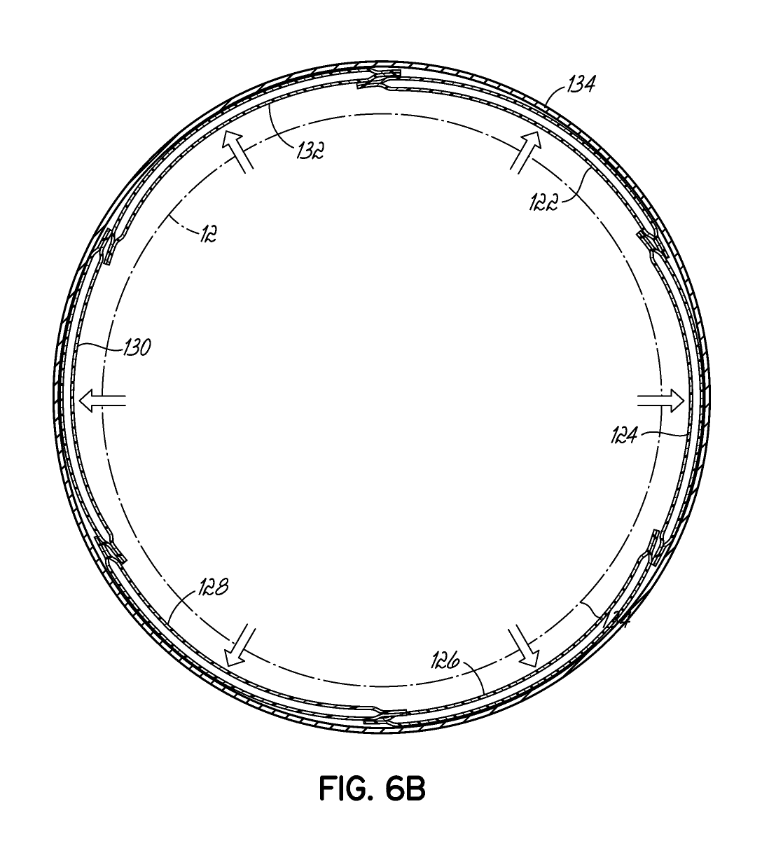

In the embodiment depicted in FIG. 5, there are limited regions in which there is no pad contact with the individual, such as at the expansion/compression regions 116, 118. To allow expansion but to ensure that all regions have coverage with the cooling and/or heating pads 122, 124, 126, 128, 130, 132 for therapeutical or other purposes, pads 122, 124, 126, 128, 130, 132 may be arranged as shown in FIG. 6A. Here, the pads 122, 124, 126, 128, 130, 132 are shown overlapping at their ends, in cross section, and may be sandwiched between two layers of elastic, compression material 134 (only one shown). This configuration allows a single garment to accommodate a variety of users of different sizes and shapes. While the concept of having one or more overlapping regions of the pads 122, 124, 126, 128, 130, 132 is shown in the context of a tubular structure, for accommodating an arm, a leg or the torso of an individual, it may also be incorporated into other designs, such as a shoulder cooling/heating/compression device, in which the shape is not necessarily tubular.

FIG. 6B shows an expanded condition of the garment or device shown in FIG. 6A. In the expanded condition, the overlap between the pads 122, 124, 126, 128, 130, 132 is reduced and yet the larger sized person will still have pads in full contact with all desired areas of the body.

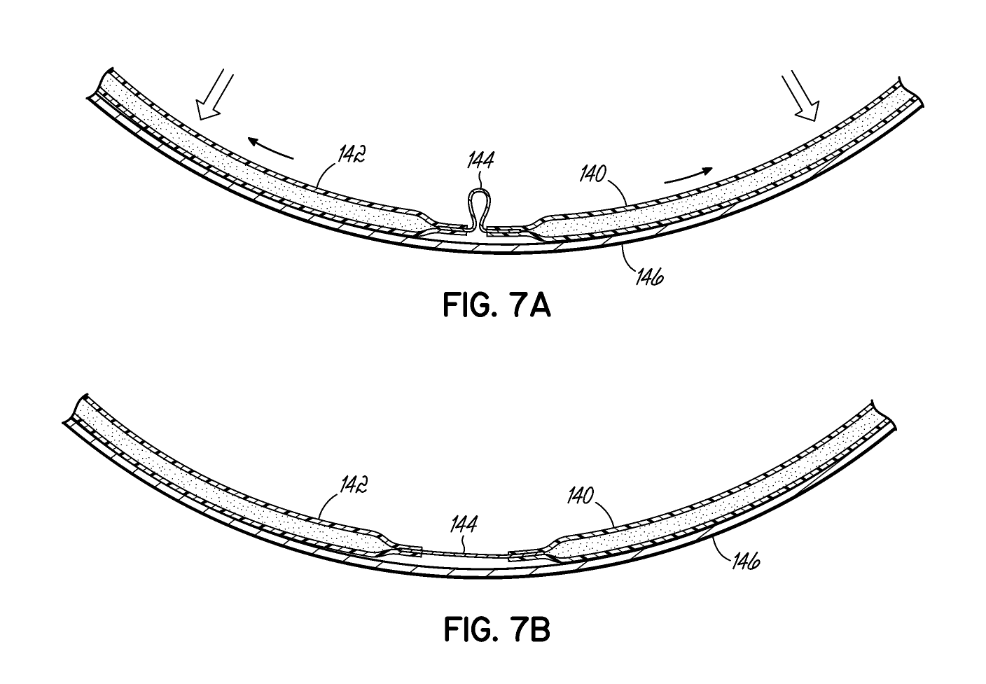

FIGS. 7A and 7B are enlarged cross sectional views showing a detail of a junction between thermal pads 140, 142 that allows for expansion. Here, the margins of the pads are attached to an expandable material 144 that will stretch and compress to adapt to different sizes of individuals and yet maintain good pad contact and compression of the body part(s) under the force of an elastic fabric 146.

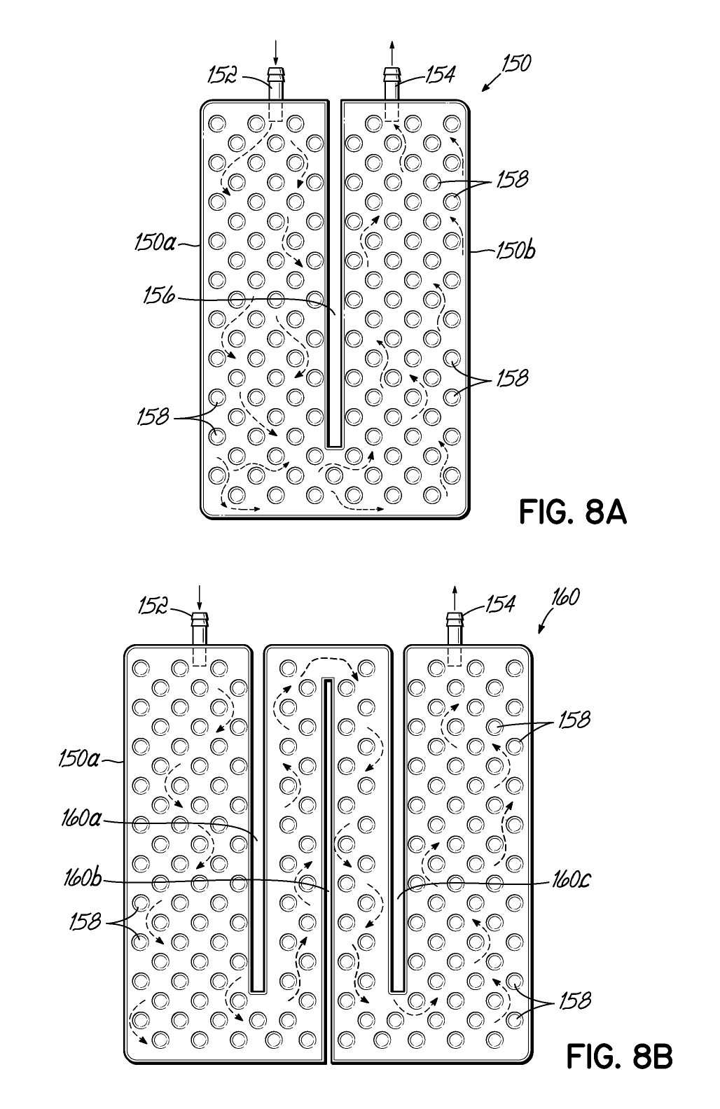

FIG. 8A illustrates an exemplary or illustrative thermal pad 150 having an inlet coupling 152 and an outlet fluid coupling 154. The pad 150 includes a gap or elongate opening 156 between the inflow and outflow sides 150a, 150b that creates a flow pattern forcing fluid to travel down one side of the pad 150 and up an opposite side of the pad 150. Additional flow diverters 158 are shown to ensure more even flow through the pad 150. The opening 156 in the pad 150 allows for expansion and contraction of the pad 150 in three dimensions so that the pad can adapt to different sizes of individual users. Flow patterns may be created by closing off areas of the pad 150 or by actually separating the pad 150 into segments. The pads 150 are preferably formed by fusing or otherwise securing two layers of a plastic material together in facing engagement, with the fused or secured areas 158 directing the flow as generally shown by the arrows. The diverters 158, shown here as circular fused or welded areas, bring fluid into contact with all areas of the pad 150. The diverters 158 may be any shape that results in the desired flow to all effective areas of the pad 150.

FIG. 8B illustrates another thermal pad 160 showing how additional openings 160a, 160b, 160c, e.g., slits, in the pad 160 may be useful for creating a more precise fluid flow pattern. This can additionally ensure good flow and cooling and/or heating to all areas of the pad 160, as well as additional freedom for expansion and conformity to different shapes and sizes of individual users. The use of compression material and breaks or slit-like openings or separations 160a, 160b, 160c in the pads 160 also allows for high compression on a variety of different users.

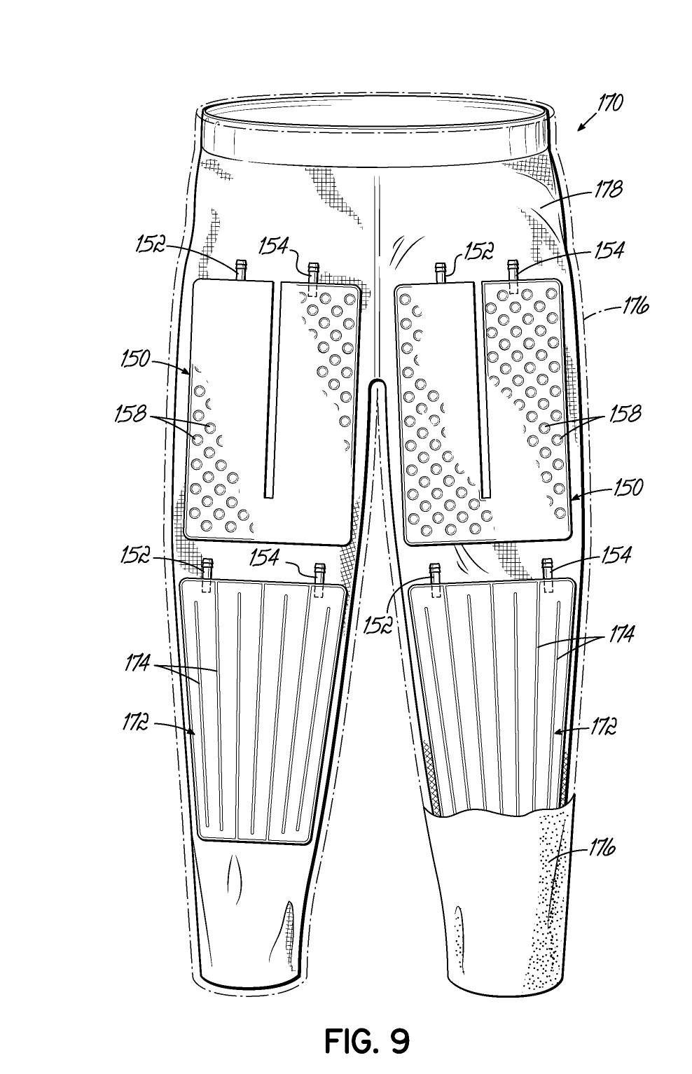

FIG. 9 is a rear view of a full length lower body compression garment 170. In this embodiment, separate pads 150, 172 are illustrated to cover different areas of the body, including upper thigh and gluteal/buttock regions, lower legs, hips, etc. Pads 172 are constructed to have lengthwise flow diverters 174. Any combination of multiple pads 150, 172 may be used, including any desired sizes and shapes. Respective inflow and outflow fluid couplings 152, 154 are shown for each pad 150, 172, while additional conduits that may be necessary to couple the pads 150, 172 to a control heating/cooling unit or console are not shown, but would be provided as understood from other disclosure herein. This garment 170, as with all other embodiments, may include any of the other features already discussed herein or to be discussed below. An outer compressive shell 176 as well as an inner liner 178 may be provided.

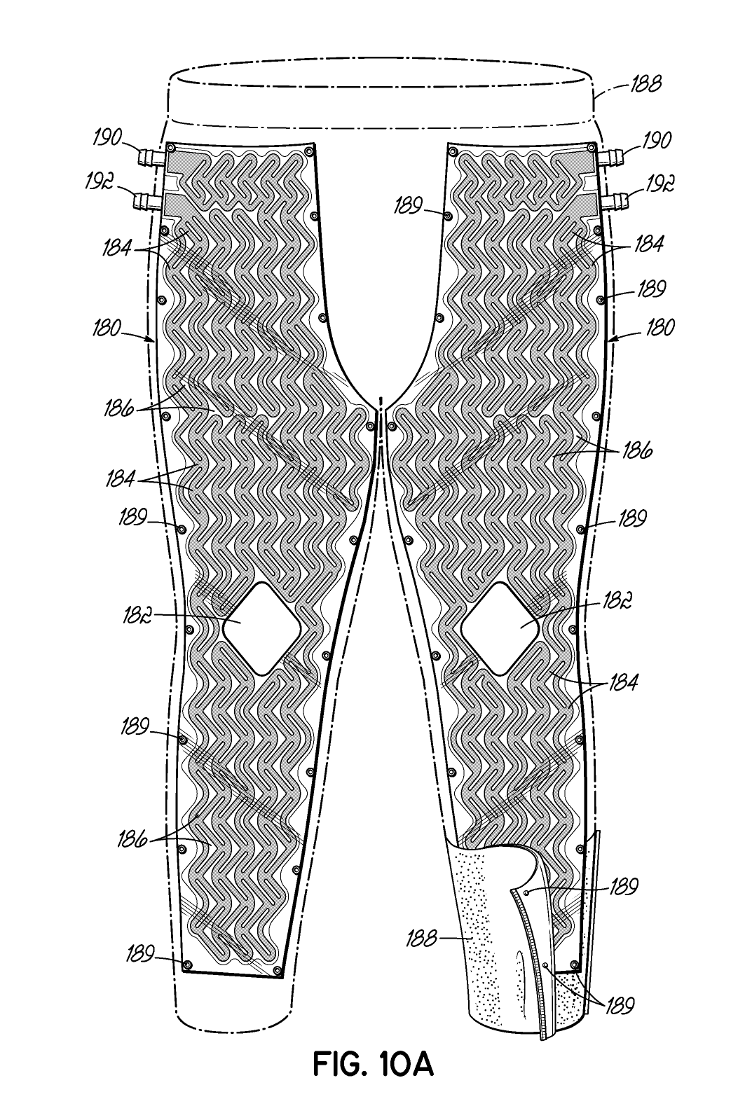

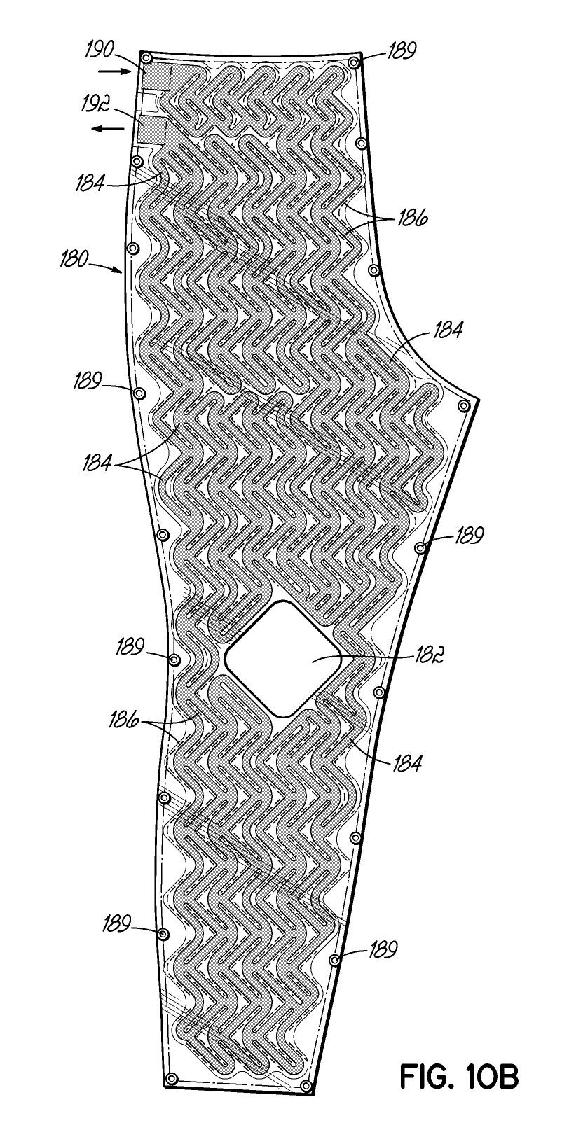

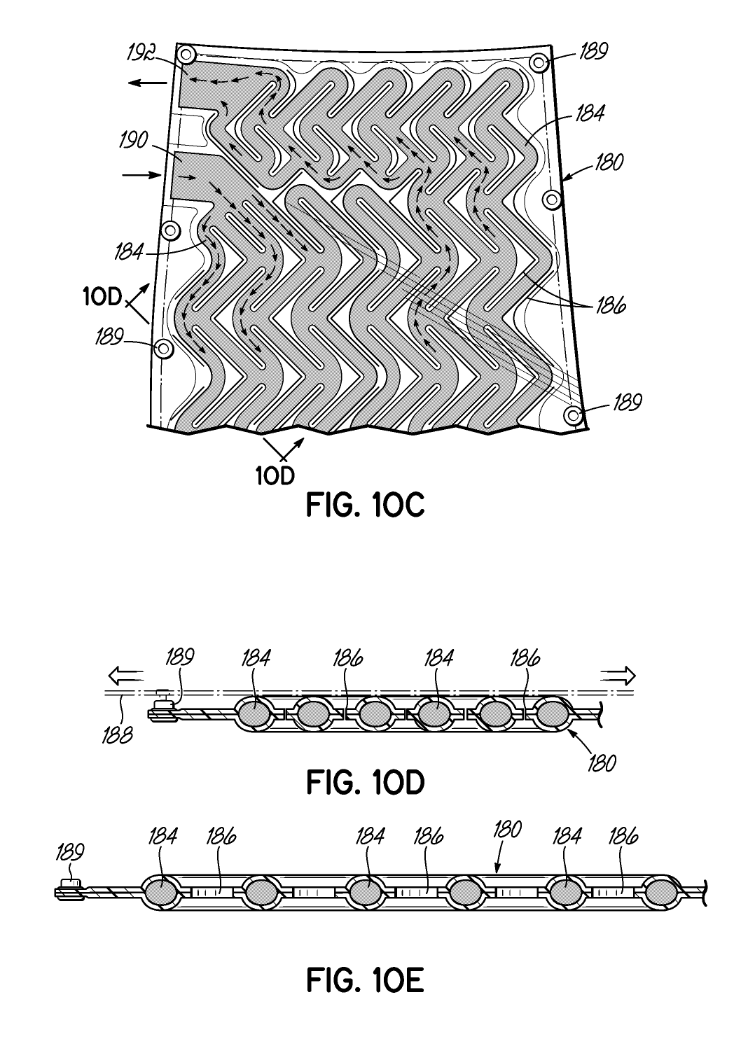

FIGS. 10A through 10E illustrate another embodiment of cooling and/or heating pads 180 constructed in accordance with the invention. Here, as best shown in FIG. 10A, each heating and/or cooling pad 180 may be constructed to extend the full length of a person's lower body. In this figure, pads 180 for the rear of the lower body are shown with cut-outs 182 at the knee region of the user for fit, flexibility and mobility purposes. The design of these thermal pads 180 includes a back-and-forth or zigzag pattern of the cooling and/or heating fluid passages 184. Slits or elongate openings 186 are provided in the pads 180 for purposes of allowing and providing for multi-directional expansion as illustrated best in FIGS. 10D and 10E. The zigzag pattern of the fluid passages 184, coupled with the similarly oriented slits 186 that also follow a zigzag pattern, provides for expansion in length, width as well as directions that are differently oriented (e.g., diagonally) between the length and the width as illustrated in FIG. 10C. It will be understood that the pads 180, again, may be formed of any desired size and shape and number. Alternatively, the pad may be formed in the shape of the entire desired garment 188 itself and, therefore, only one pad would be necessary to envelop the desired area of the individual user. The pad(s) 180 may be snapped or otherwise fastened to the other portions of the garment 180 using fasteners 189. Respective inlet and outlet fluid couplings. 190, 192 are provided on each pad 180.

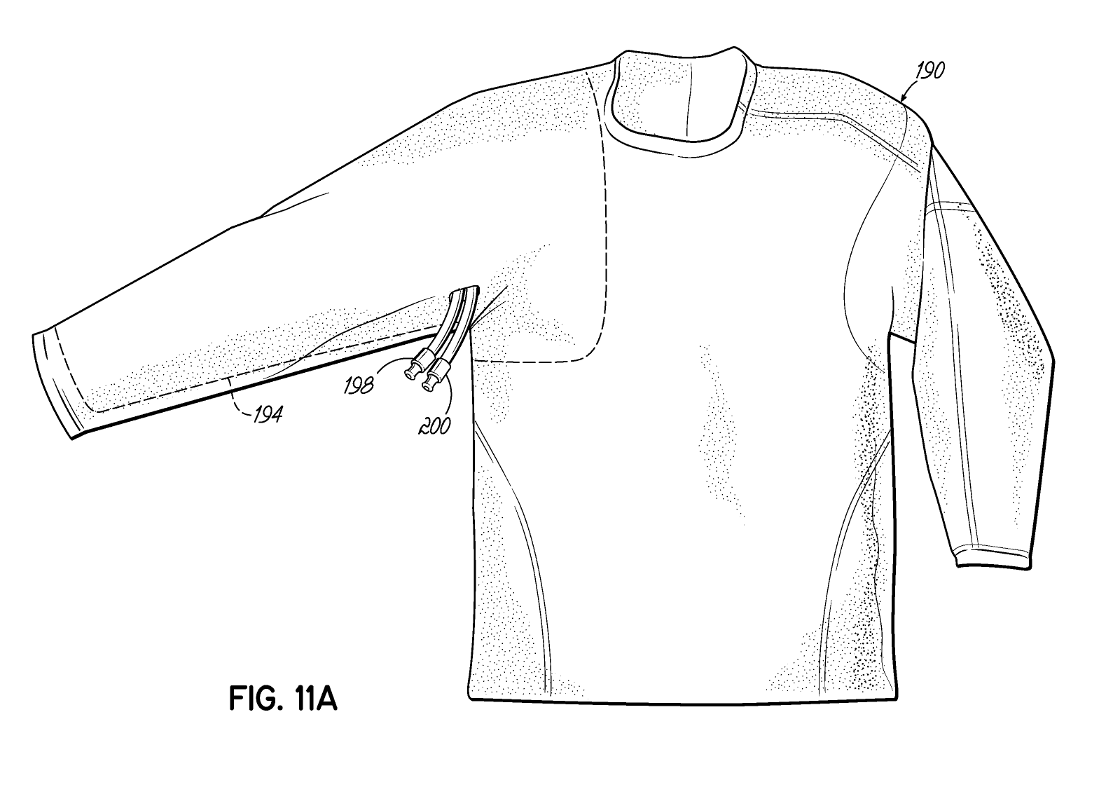

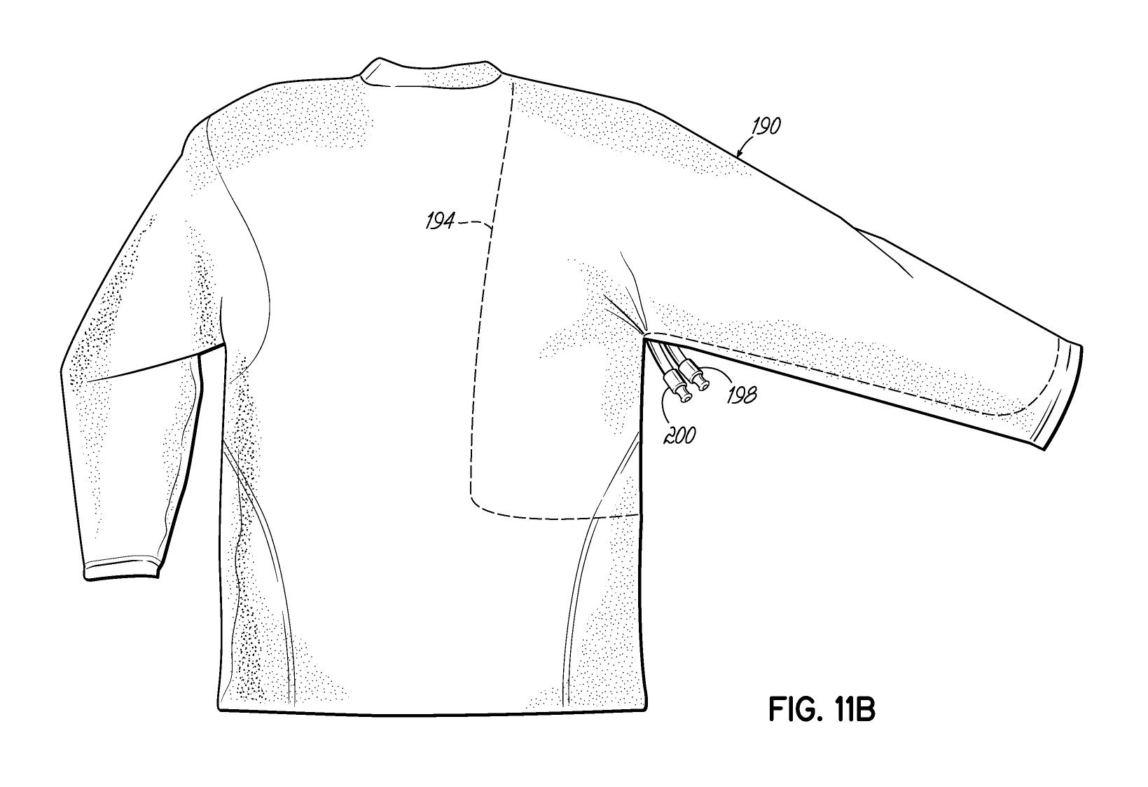

FIGS. 11A and 11B respectively illustrate the front and rear of a cooling and/or heating compression garment 190 with a thermal pad 194 shaped and constructed to provide therapy to a shoulder, back and arm region of the individual. As previously described, respective inlet and outlet fluid couplings 198, 200 are provided for reasons described herein. Such garments 190 may be constructed for either side of the patient's body and with any of the features described herein.

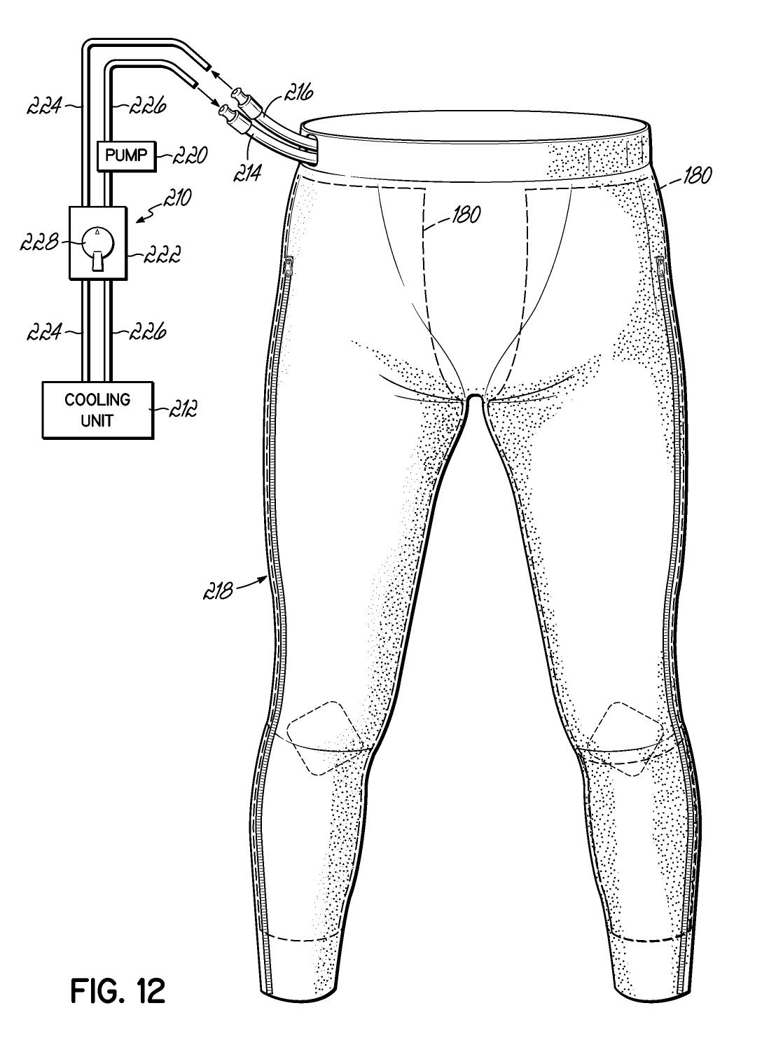

FIG. 12 is a first illustrative embodiment of a recirculation cooling system 210 that assists with cooling temperature control. Generally, the system 210 includes a cooling unit 212, respective input and output conduits 214, 216 as previously discussed, a desired compression/cooling garment which in this embodiment is shown as a pair of compression pants, a pump 220, and a flow control device 222. It will be appreciated that the pump 220 and/or the control device 222 may be incorporated into the cooling unit 212 such that a single control unit or console is provided and includes all of the necessary fluid and control components. The flow control device 222, as will be further understood below, may be designed with varying levels of complexity depending on the amount of temperature control desired, for example. It will be best to place the pump 220 downstream of the flow control device 222 to ensure proper recirculation. The garment 218 may be constructed in any manner described herein. In this embodiment, the flow control device 222 may comprise a variable valve or a static shunt that will redirect a particular amount of the return fluid from the return or outlet conduit 224 back into the supply fluid conduit 226. The amount of redirected fluid will be controlled by a suitable dial 228 or other mechanism in the case of an adjustable flow control device 222 such as a valve. Therefore, in general, the flow control device 222 provides for a recirculation path that adds slightly warmer fluid to the cold thermal fluid being pumped into and through the supply conduit 226. As one example, the cold fluid coming from the cooling unit 212 may exit the cooling unit 212 at approximately 2°-3° C. and this may be too cold for various individuals and/or various therapies or uses. Recirculating and adding return fluid, which has been warmed by the body of the individual user as it flows through the compression garment 218, effectively adds warm fluid to the cold fluid via the control device 222 or another suitable recirculation passage thereby resulting in an elevation of the temperature of the input fluid. For example, a more desirable temperature such as 8°-10° C. may be achieved for the fluid entering the suit 218. As will be understood from the variations disclosed below, many different recirculation systems may be used after gaining an understanding from the present disclosure.

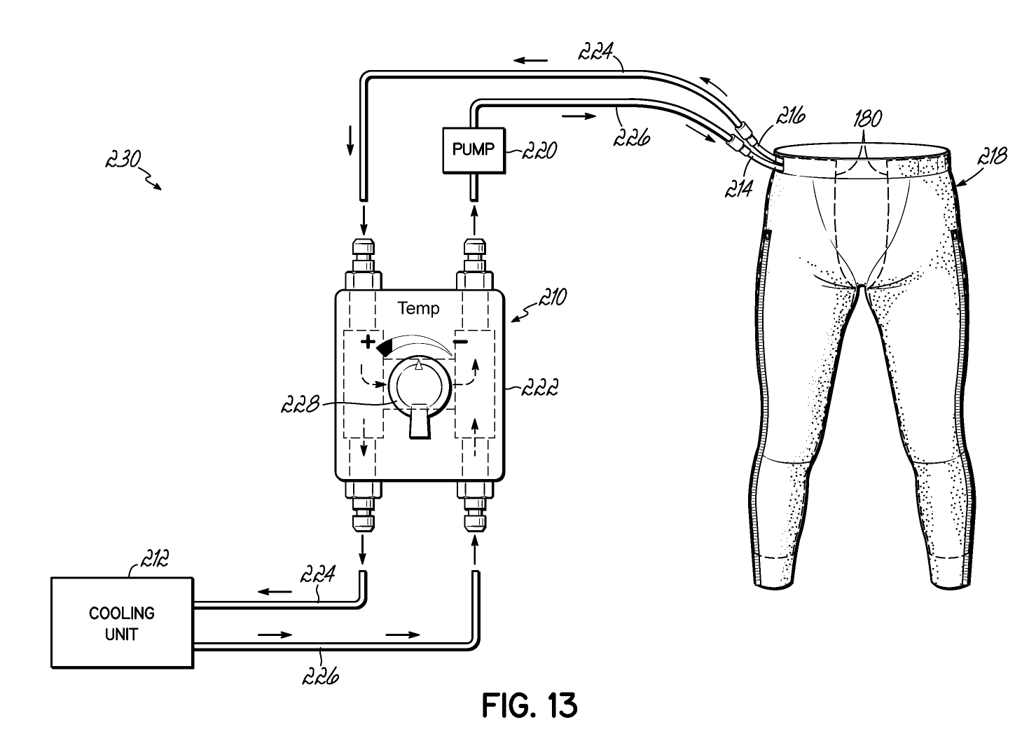

FIG. 13 illustrates a system 230 similar to the system 210 shown in FIG. 12, however additional detail is provided for the flow control device 222. The flow control device 222 comprises a variable flow control valve which may be rotated as shown to raise or lower the temperature of the input fluid. Turning the dial 228 in a clockwise direction will raise the temperature of the input fluid, by adding a selected amount of warmer fluid from the return conduit 224 to the input conduit 226 as shown by the dashed arrows, while rotating the dial 228 in a counterclockwise direction will lower the temperature of the input fluid until a minimum temperature is reached, i.e., the temperature of the input fluid arriving from the cooling unit 212 and pump 220. Any return fluid that is not recirculated into the input conduit 226 is returned to the cooling unit 212, for example.

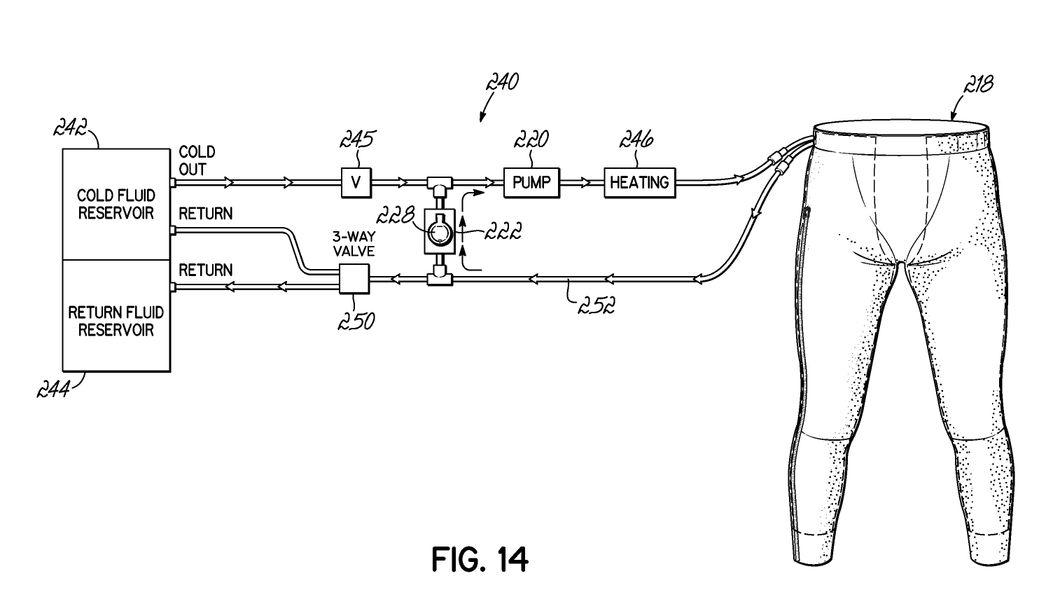

FIG. 14 illustrates another system 240 in which a cold fluid reservoir 242, as well as a return fluid reservoir 244 are provided. As previously mentioned, the fluid may be cooled in various manners, including through the use of thermoelectric cooling (e.g., Peltier) devices, or other cooling or refrigeration systems, or simply by using an insulated cooler filled with ice and water. The cooled fluid is pumped out of the cold fluid reservoir 242 through a shut-off valve 245 by a pump 220 and into the compression garment 218 as described herein. Optionally, a heating device 246 may be thermally associated with the input fluid, such as schematically shown, to optionally provide for heating of the fluid as described herein. It will be appreciated that the heating device 246, as well as the pump 220 and other components may be located in any suitable area of the system 240 for achieving the results as described. Location in a single console or control until will often be optimal. A flow control device 222 is provided in a recirculation path for purposes as described in connection with FIGS. 12 and 13. In this embodiment, a three-way valve 250 is placed in the return or output flow path 252 and may be adjusted by the user to either return the fluid to the return fluid reservoir 244 or to the cold fluid reservoir 242. Returning the fluid to the cold fluid reservoir 242 will result in the ability to provide longer therapy sessions, but with gradually increasing temperatures in the case of using a cooler full of ice and water which will gradually be warmed by the fluid returning from the compression garment 218. The return of fluid to a separate return fluid reservoir 244 obviates this problem, but gradually depletes the fluid in the cold fluid reservoir 242 resulting in shorter therapy sessions.

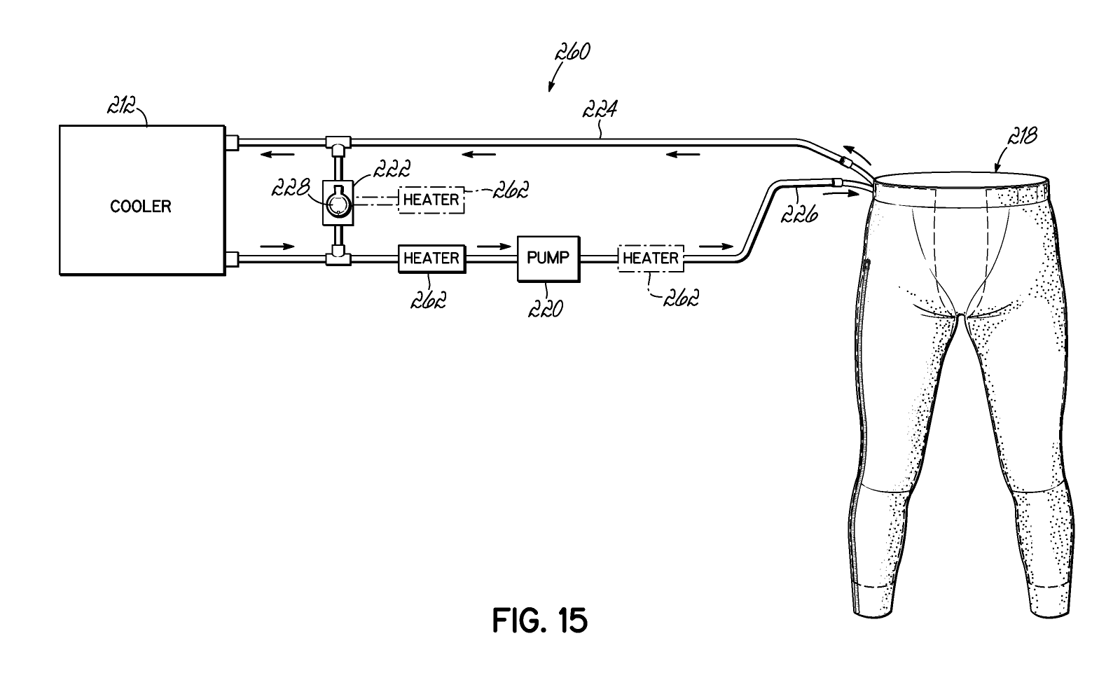

FIG. 15 shows another alternative system 260 with a heater 262 optionally placed in thermal communication with the flow control device 222. This may be accomplished in any suitable manner and, as the fluid recycles from the compression garment 218, the fluid is slightly warmed to a more physiologic level of cooling. That is, if recycling the return fluid itself does not raise the temperature of the input fluid to the desired level and/or within a desired time frame, then additional heat may be added with a suitable heater 262, such as the heater 262 placed in thermal communication with the flow control device. Other locations for the heater 262 may be provided instead, such as shown in FIG. 15 in dash-dot as well as solid lines.

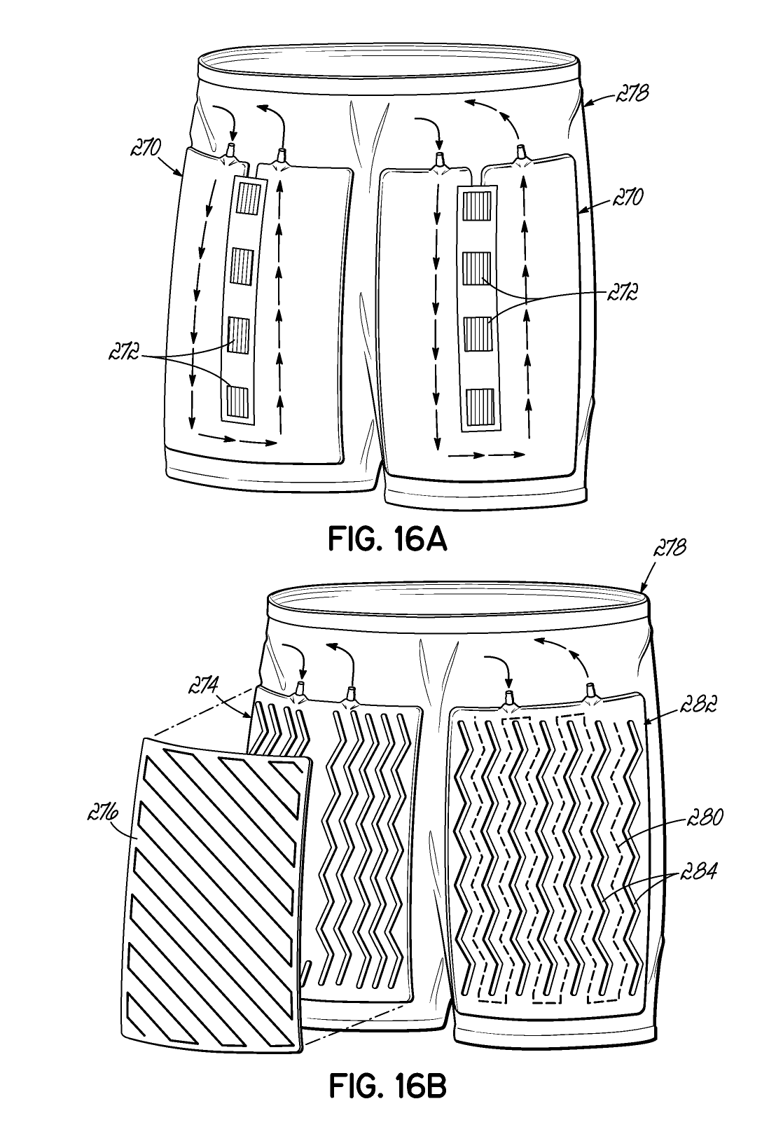

FIGS. 16A and 16B illustrate garments that have heating/cooling pad structures of alternative designs. In FIG. 16A, the pads 270 include both cooling channels as schematically shown by the arrows to direct cooling fluid through the pad 270 in a downward and then upward flow pattern as previously described, as well as electric heating elements 272 for alternately or also providing selective heat therapy to the user. This allows for contrast therapy, and other therapies or uses as discussed herein. For example, contrast therapy will allow the individual to cycle heating and cooling to the desired areas of the body, depending on the particular item of apparel being worn by the user. The electric heating elements 272 may be integrated into the pads 270 in various manners, with the embodiment of FIG. 16A utilizing electric heating elements 272 that may be secured directly to the pads 270, such as through the use of adhesive. FIG. 16B illustrates two additional alternatives for applying heat and cooling therapy to the user. On the left side of the garment a cooling pad 274 is shown, and may be constructed in any of the various manners described herein, as examples, and an electric heating mesh structure 276 is applied directly on top of the cooling pad 274 and secured thereto in a suitable manner. This may be in any of the manners described herein, with some examples being fasteners that allow removal of the heater mesh 276, or through the use of an outer compression layer of fabric (not shown) that maintains the heater mesh 276 in position on the individual. Alternatively, the heater mesh 276 may be located between the cooling pad 274 and the inner liner 278 of the garment shown in FIG. 16B such that the heat is applied closer to the skin of the individual user. On the right side of FIG. 16B another alternative is shown in which a heater mesh 280 is integrated directly into the cooling pad 282 between the cooling channels 284.

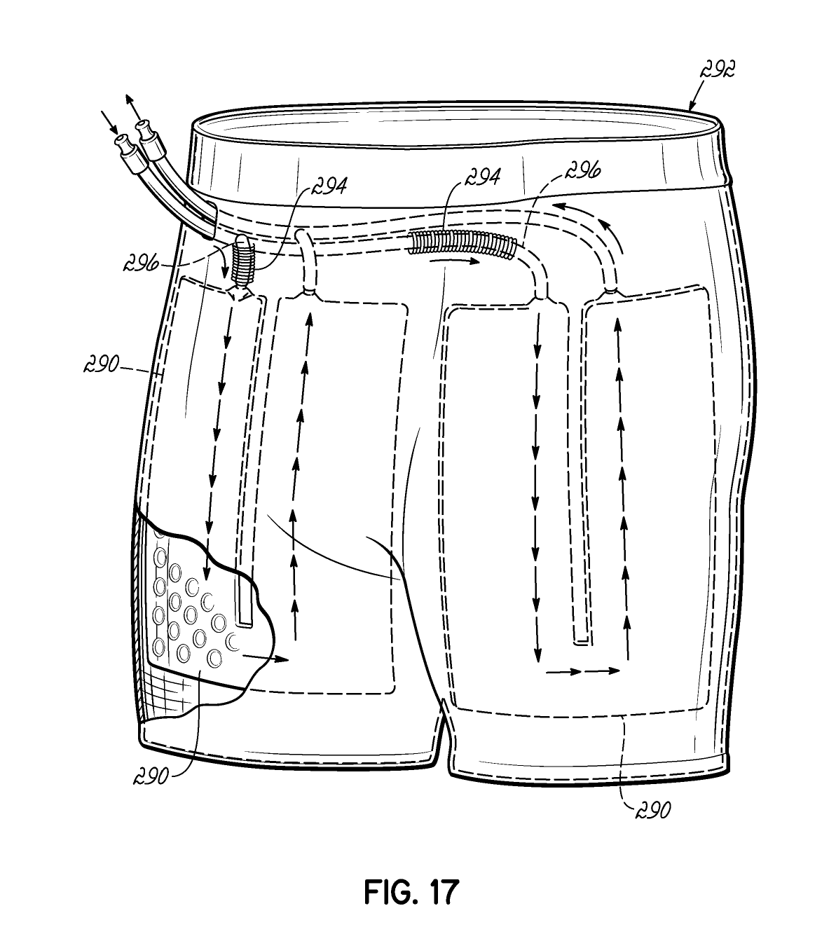

FIG. 17 illustrates a pair of heating pads 290 integrated into or otherwise associated with a compression garment 292, which may be formed in any of the various manners described, and also including integrated electric heaters 294 placed in thermal contact with the internal input thermal fluid conduit structure 296 within the garment 292. This is another manner of providing heat to fluid being directed into the pads 290 for any desired purpose(s).

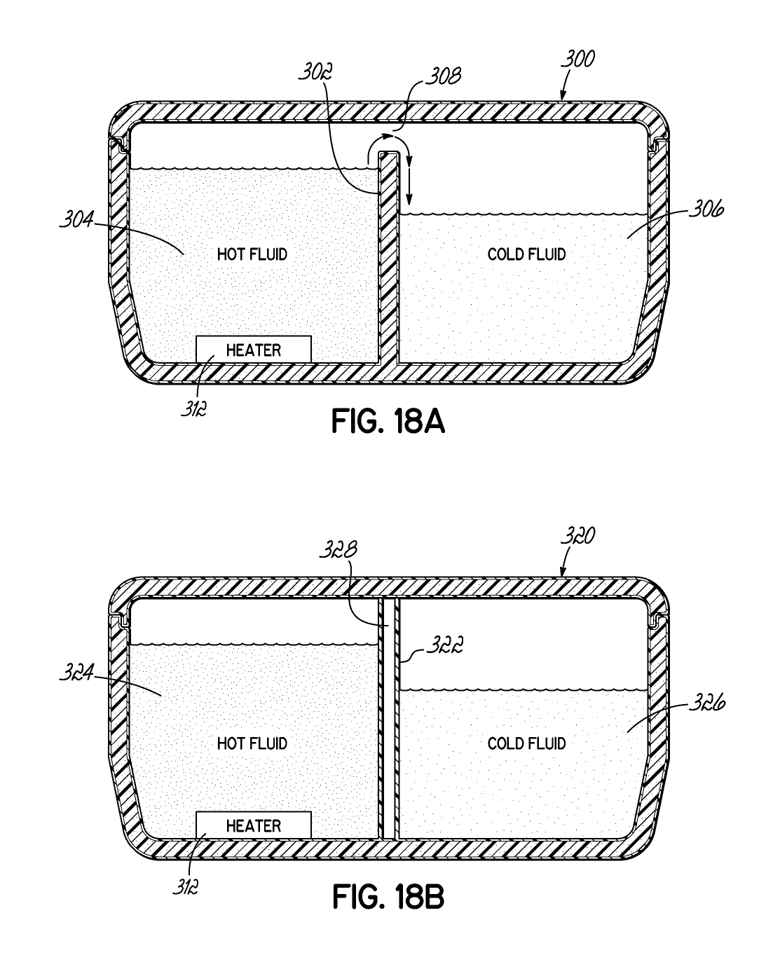

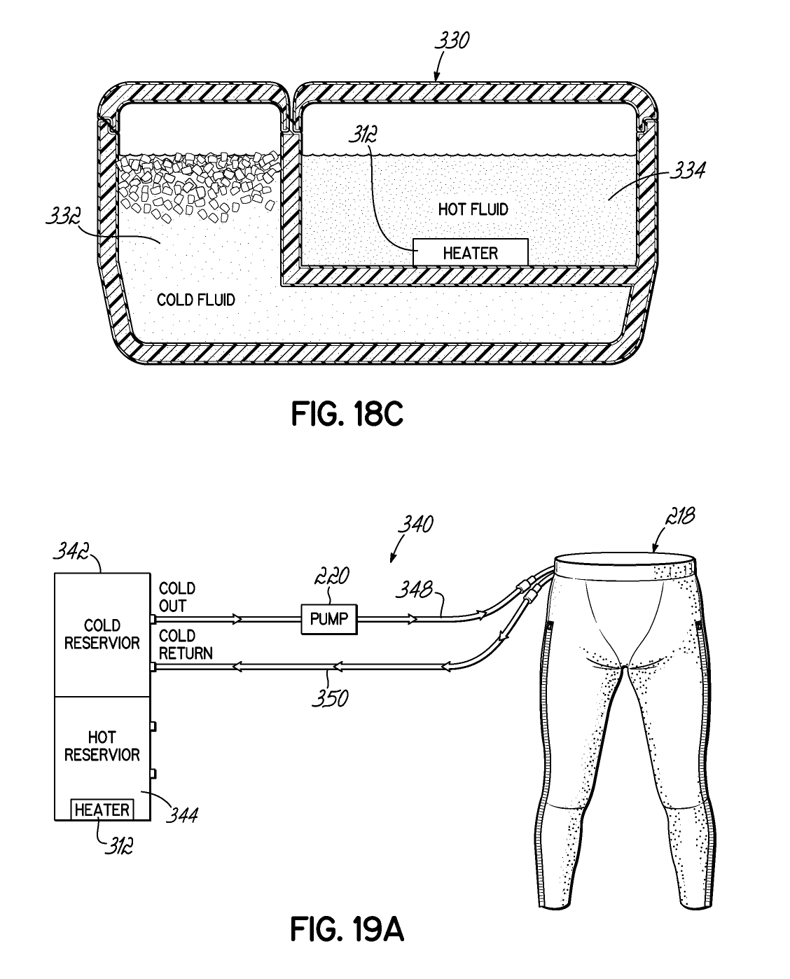

FIGS. 18A, 18B and 18C illustrate additional alternative embodiments for use with therapies involving heating and cooling of the body. These thermally insulated containers include separate internal chambers for containing the hot fluid and the cold fluid. The thermally insulated container 300 of FIG. 18A includes an insulating wall 302 between two chambers 304, 306 having an upper space or opening 308 allowing hot fluid to overflow into the cold fluid chamber 306 to prevent spillage. This is useful when the hot fluid chamber 304 is being filled with recirculated fluid from a compression garment as discussed herein. The hot fluid chamber 304 may include a heater 312 for actively heating the fluid. The thermally insulated container 320 of FIG. 18B similarly includes an insulating dividing wall 322 between two chambers 324, 326, however, this dividing wall 322 provides for no communication between the chambers 324, 326. The dividing wall 322 of FIG. 18B is thermally insulating in nature by including an air insulating space 328 therein. FIG. 18C illustrates a two chamber thermally insulating container 330 including a cold fluid chamber 332 and a hot fluid chamber 334 in which the two chambers 332, 334 are stacked upon one another.

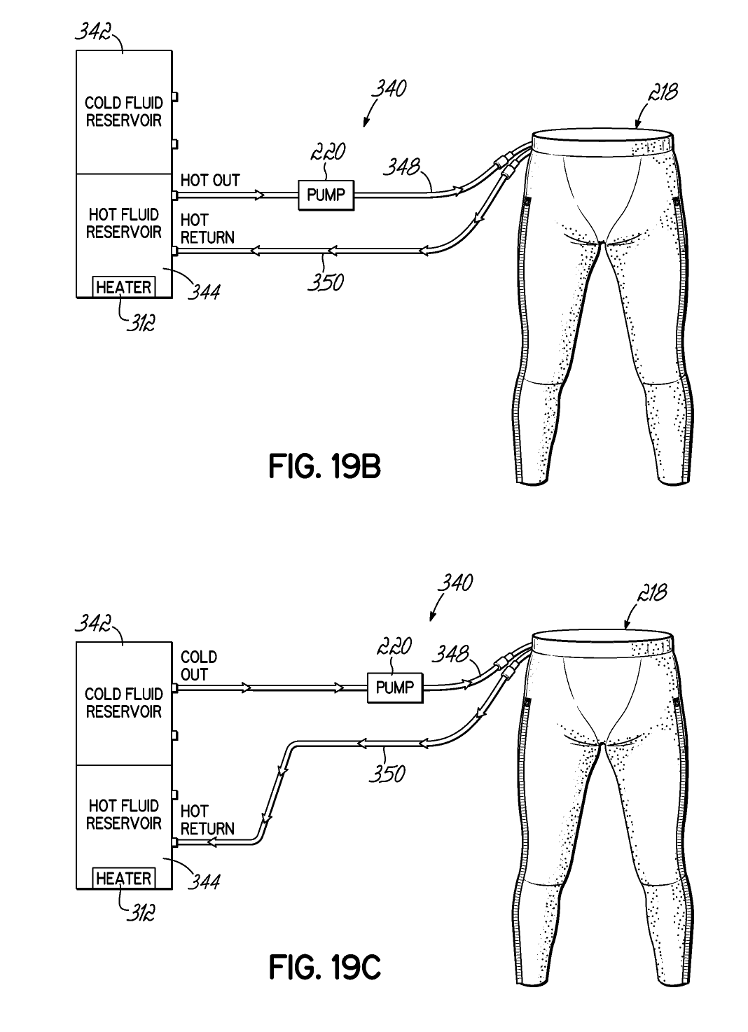

FIG. 19A illustrates a system 340 with a compression garment 218 and respective cold and hot fluid reservoirs 342, 344. FIGS. 19B and 19C illustrate the same system 340 with different connections being made based on the desired therapy or effect. The cold fluid reservoir 342 may be constructed in any manner disclosed herein, or in other manners, and the hot fluid reservoir may, for example, include a heater 312. The heated reservoirs associated with the invention may take on any desired form. As one option, the temperature of the fluid in the heated reservoir 312 may be regulated with a radiator having a suitable temperature sensor to prevent the fluid from getting too hot. Other sensor-based temperature controls may be used instead. In FIG. 19A the cold reservoir 342 is connected via conduits 348, 350 to the compression garment 218 to provide cold fluid, such as ice water, via the pump 220 to the compression garment 218 and allowing return of the fluid to the cold reservoir 342. FIG. 19B, in a contrast therapy session, for example, allows the user to switch the conduits 348, 350 from the cold reservoir 342 to the hot fluid reservoir 344 thereby providing heated fluid to the compression garment 218 and a return of the heated fluid to the hot fluid reservoir 344 to provide heat therapy to the individual. FIG. 19C illustrates a short purge cycle in which cold fluid is pumped into the compression garment 218 and the fluid is returned to the hot fluid reservoir 344 where it may be heated once again if it is desired to repeat the heat therapy cycle or session as illustrated in FIG. 19B. Once the compression garment 218 is purged of the hot fluid, the cold therapy session may be initiated again.

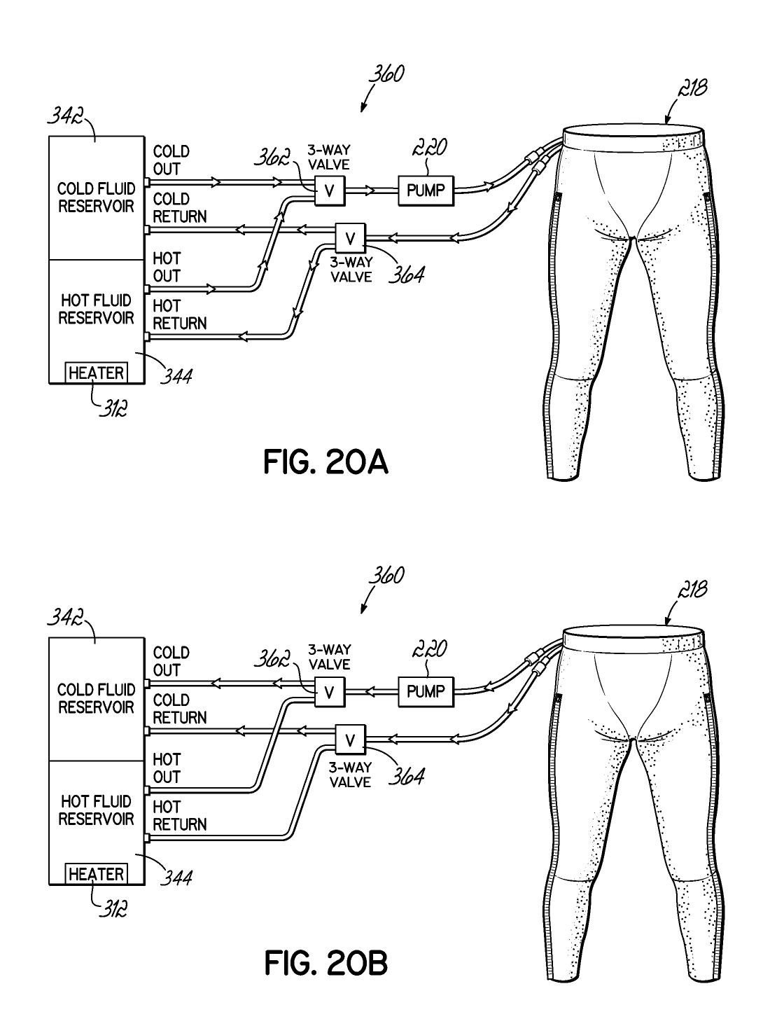

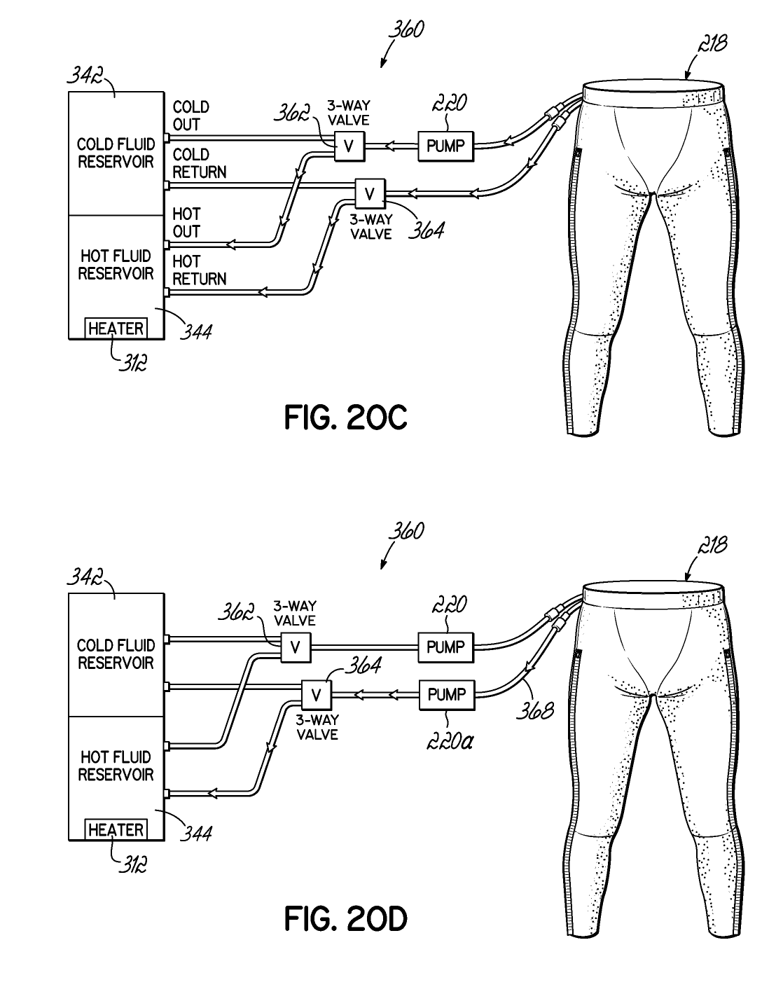

FIG. 20A illustrates another system 360 for providing contrast or any other hot/cold therapy to an individual wearing the compression garment 218 or otherwise using the pad(s) of choice. In this system, cold fluid is pumped out of the cold fluid reservoir 342 through a three-way valve 362 and into the compression garment 218. At the same time, hot fluid may be pumped out of the hot fluid reservoir 344 and directed through the three-way valve 362 to provide temperature control to the cold fluid, for example, thereby raising the temperature of the ice cold water delivered from the cold fluid reservoir 342 from a temperature of 2°-3° C. to a temperature of, e.g., 8°-10° C. as this temperature may be more comfortable for the individual user. The fluid is returned through another three-way valve 364 and may be returned to either or both of the cold fluid and hot fluid reservoirs 342, 344. At the end of a hot or cold fluid therapy session, the pump 220 is switched off allowing fluid in the compression garment 218 to return to the appropriate reservoir 342 and/or 344. FIG. 20B illustrates a purging segment in which the pump 220 is turned off and the fluid is being returned to the cold fluid reservoir 342 through the three-way valves 362, 364. FIG. 20C illustrates a purging segment in which the pump 220 is switched off and the fluid returns via the three-way valves 362, 364, switched to an alternate location, into the hot fluid reservoir 344. FIG. 20D illustrates another alternative purge cycle. In this system, a pump 220a has been added to the return conduit 368 to drain the compression garment 218 between hot and cold segments of the contrast therapy session. This is a manner of better purging the fluid channels or passages within the compression garment 218 than simply allowing the natural compression of the garment 218 to squeeze the fluid from the channels or passages back into the conduits and into one or both reservoirs 342, 344. In this embodiment, the supply pump 220 would be turned off during the purge cycle.

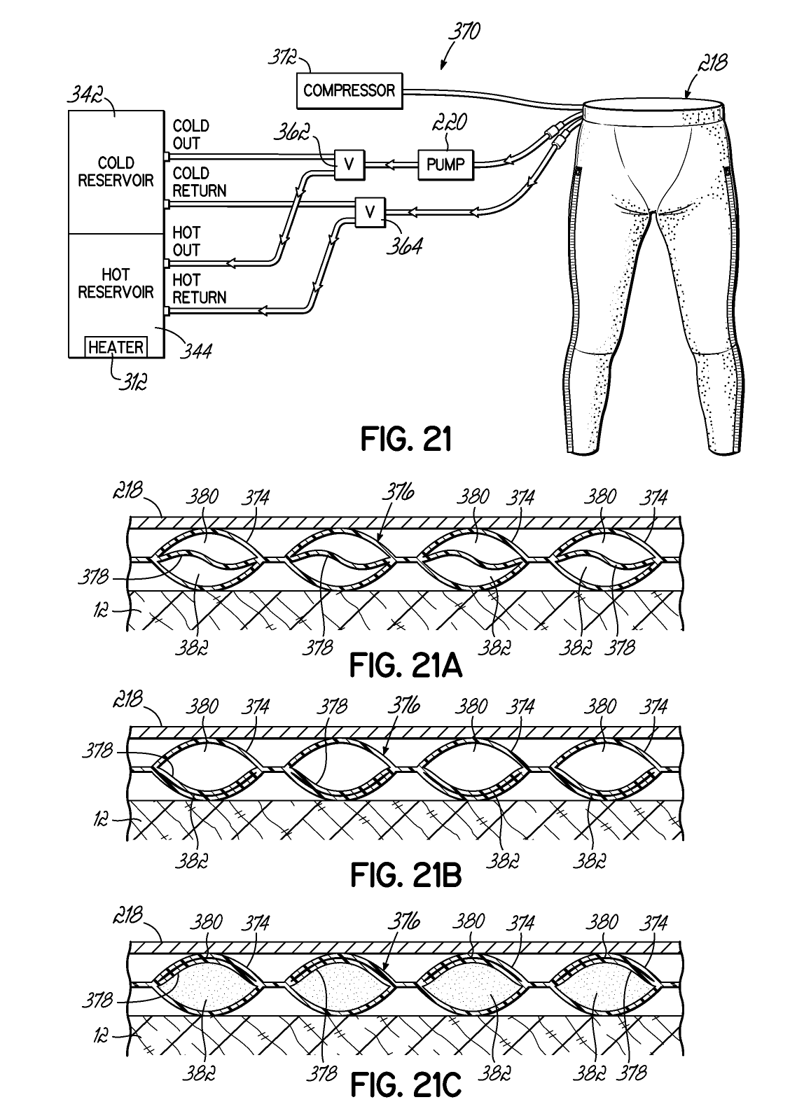

FIG. 21 illustrates another alternative system 370 and purge cycle. In this system, an air compressor 372 pumps air into the compression garment 218 between hot and cold therapy cycles to push remaining thermal fluid from the compression garment 218 back into the appropriate reservoir 342 and/or 344. This compressed air can also be used for more active compression therapy and may be applied continuously or intermittently. FIGS. 21A-21C illustrate one possible design for the fluid channels 374 within the heating/cooling pads 376 in which inner membranes 378 within the fluid channels 374 divide the channels 374 between air sections 380 and thermal fluid or liquid sections 382. In FIG. 21A air is being introduced into the channels in the air sections 380 to gradually force the liquid fluid from the fluid sections 382. Once the membranes 378 are in the positions shown in FIG. 21B, all of the thermal fluid has been forced from the channels 374. FIG. 21C illustrates the situation in which the fluid sections 382 of the channels 374 are full of thermal fluid during a heating or cooling therapy session and no pressurized air is introduced into the air sections 380. As another option, one of the sections 380 or 382 may receive a cooling fluid while the other section 380 or 382 may receive heating fluid, such as when using the system 370 in a contrast therapy method, as described herein.

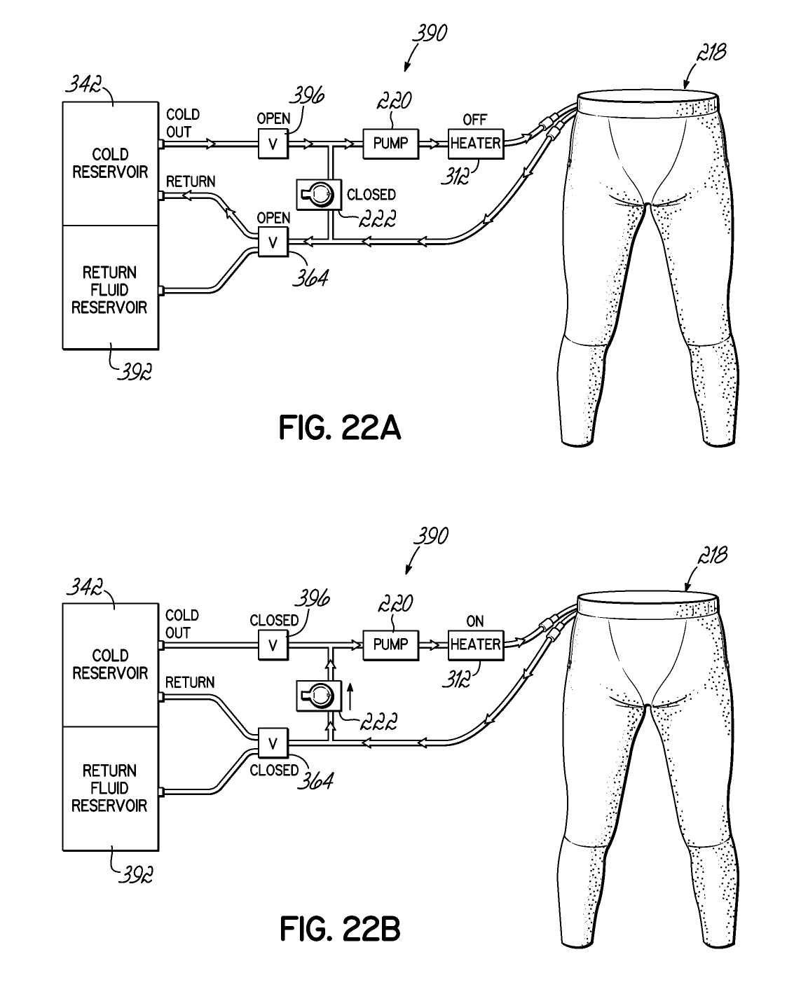

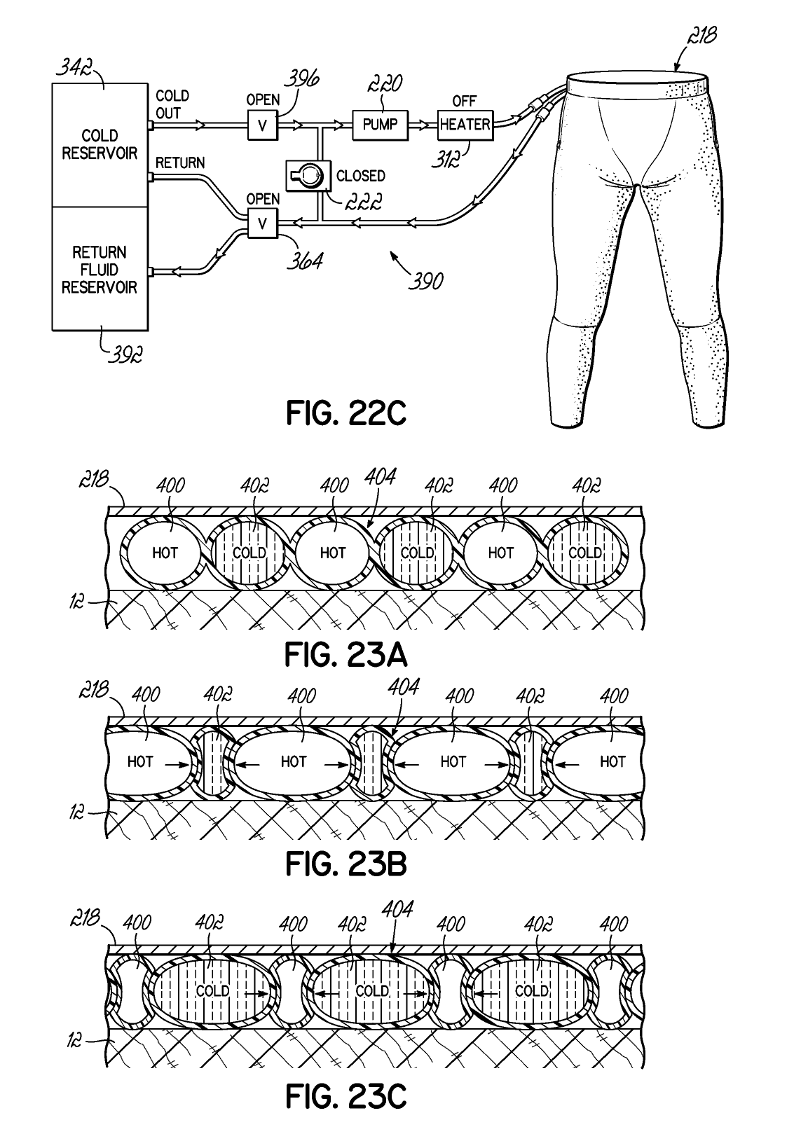

FIGS. 22A, 22B and 22C illustrate another system 390 including a cold reservoir 342 and a return fluid reservoir 392. In this system 390 there need not be any heater in the return fluid reservoir 392. Instead, a heater 312 is located on the fluid input side of the system 390, such as by being placed within the input fluid path as shown. In FIG. 22A, the heater 312 is turned off and a cold therapy session is in progress by pumping cold water from the cold reservoir 342 into the compression garment 218 and returning the water to the cold reservoir 342 through the three-way valve 364 which is set to a position to return the water into the cold reservoir 342. In FIG. 22B, the three-way valve 364 is completely closed and an input valve 396 is also closed, while the flow control device 222 in a recirculation path is open to recirculate the fluid from the compression garment 218 through the pump 220 and heater 312 for conducting a heat therapy session. FIG. 22C illustrates the three-way output valve 364 open and in a position to direct return fluid into the return fluid reservoir 392. The flow control device or recirculation valve 222 is completely closed, while the input valve 396 is open. The heater 312 is off and, therefore, a cold therapy session is in progress by pumping cold fluid from the cold reservoir 342 into the compression garment 218 and then returning that fluid from the compression garment 218 after being cycled through the garment 218 and delivering the return fluid into the reservoir 392 through the opened three-way valve 364.

FIG. 22C illustrates a short purge cycle after a heat therapy session, after which the cold therapy session may begin again.

FIGS. 23A, 23B and 23C illustrate an alternative channel design for the heating/cooling pads. In this design, alternating or different channels 400, 402 contain hot and cold fluids. In this particular example, the hot and cold fluids alternate with every other channel 400, 402 containing hot or cold fluid. Other designs are possible as well. One advantage of this design is that when the hot fluid is pumped into the heating/cooling pad 404, the hot fluid channels 400 will expand and compress the cold fluid channels 402 thereby purging the cold fluid from the pad 404. As shown in FIG. 23C, the same effect will occur when introducing pressurized cold fluid into the cold fluid channels 402. This will compress the hot fluid channels 400 and purge the hot fluid from the pad 404.

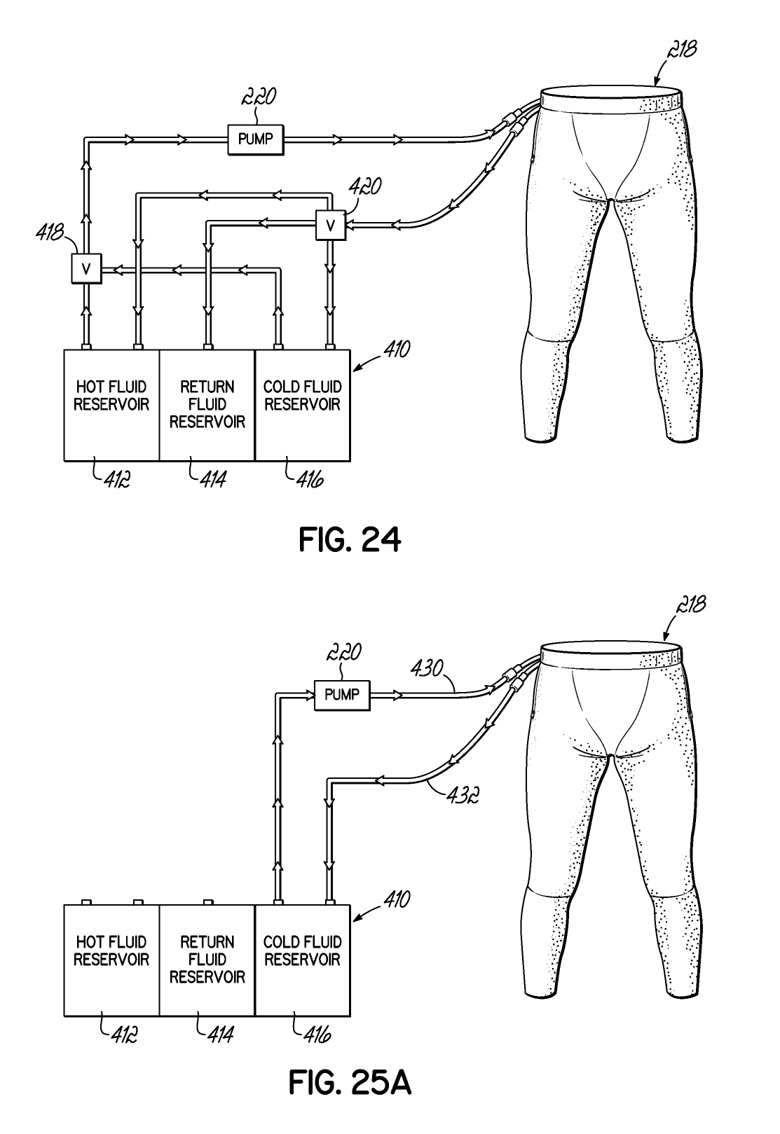

FIG. 24 illustrates a three chamber thermally insulated container 410 including a hot fluid reservoir 412, a return fluid reservoir 414 and a cold fluid reservoir 416. Each may be constructed in any suitable manner such as elsewhere described herein. This allows for contrast hot/cold therapy sessions as well. As shown in FIG. 24, hot fluid may be pumped from the hot fluid reservoir 412 through a valve 418 and into the compression garment 218 and then returned through another valve 420 into one or both of the hot fluid and/or return fluid reservoirs 412, 414.

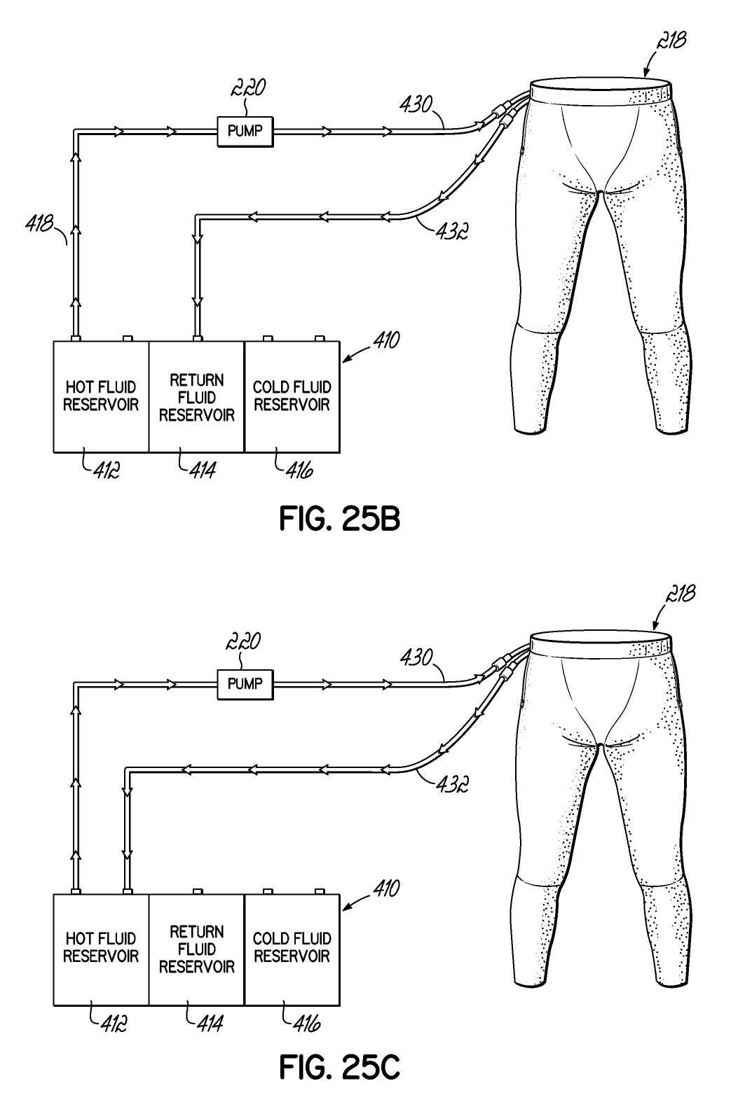

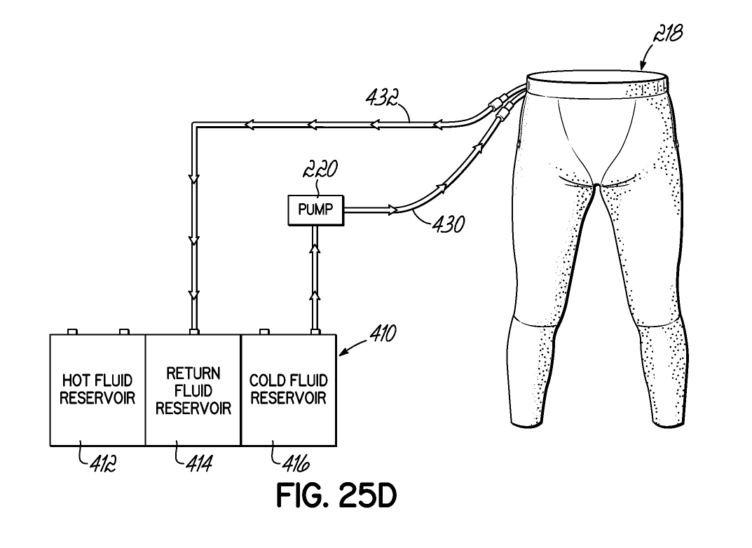

FIG. 25A Illustrates that the three chamber insulated container 40 may be connected such that an input conduit 430 and pump 220 are connected to the cold fluid reservoir 416 and the fluid is returned to the cold fluid reservoir 416 from the compression garment 218 through a conduit 432 during a cold therapy session. FIG. 25B illustrates that during a hot therapy session, hot fluid may be pumped from the hot fluid reservoir 412 into the compression garment 218 and returned to the return fluid reservoir 414. FIG. 25C illustrates that the fluid may be alternatively returned back to the hot fluid reservoir 412 where it may be reheated. FIG. 25D illustrates pumping cold fluid from the cold fluid reservoir 416 during a cold therapy session, and returning the fluid to the return fluid reservoir 414 or chamber in the three chamber insulated container. It will be appreciated that in each of the embodiments, the various reservoirs may be separate units or, for compactness, may be designed as a single container.

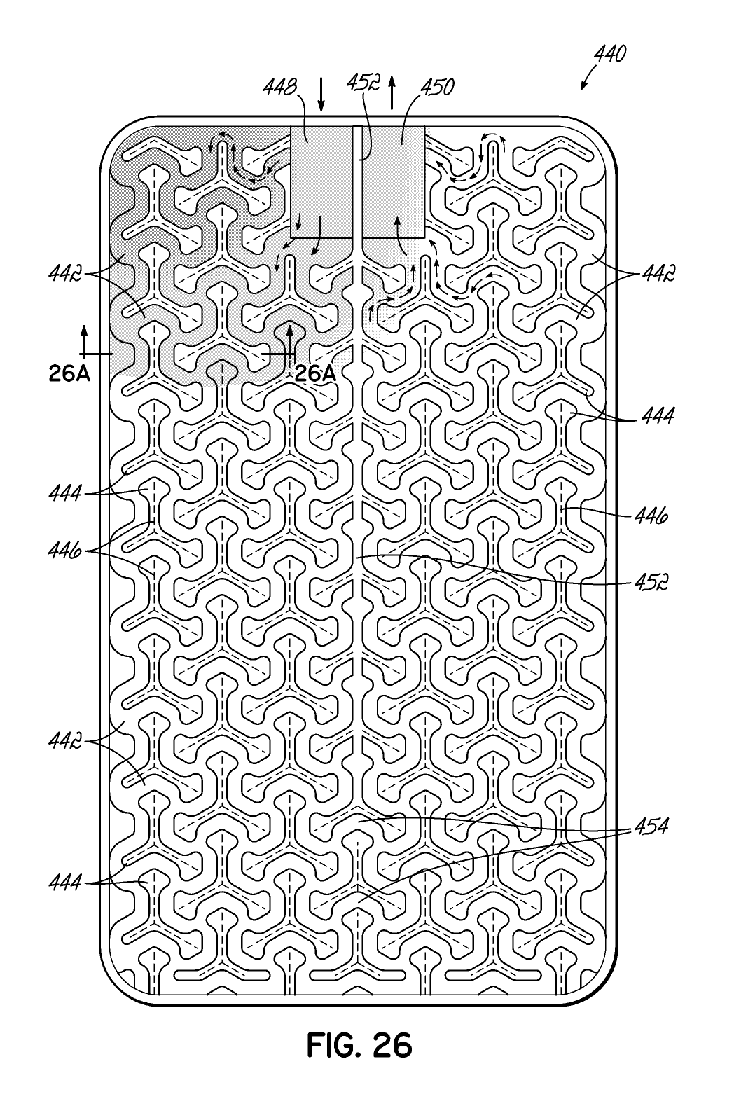

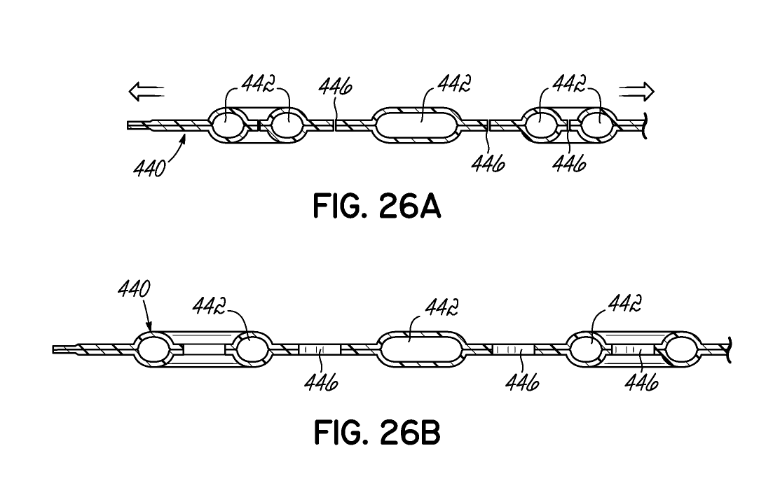

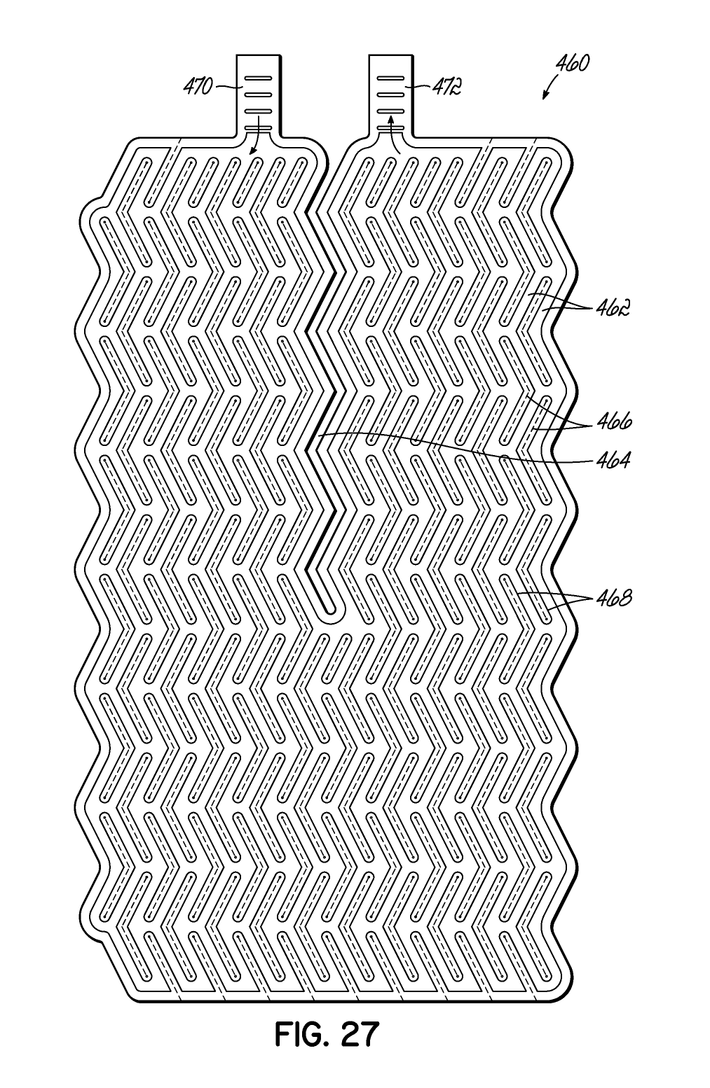

FIG. 26 is an elevational view of another alternative pad 440 including fluid channels 442 and solid (e.g., fused) pad sections 444 defining dividing elements between fluid channels 442. The solid sections 444 include slits 446 as indicated by dashed lines to allow for multi-directional (i.e., at least bi-directional) stretching or elasticity in the pad 440. More specifically, the pad 440 may be stretched in length, width, and generally diagonally or in other directions between the length and the width. This allows the pad 440 to accommodate a wider variety of individual users and their respective shapes and sizes. The pad 440 includes at least one fluid inlet 448 and one fluid outlet 450 and the general direction of flow will be down the left side of the pad 440 as viewed in FIG. 26, and up the right side as generally previously described, as a central divider 452 extends almost the full length of the pad 440 but provides cross channels 454 near the bottom of the pad 440 for communicating the fluid flow from the left side to the right side.

FIGS. 26A and 26B are respective cross sections taken along line 26A-26A of FIG. 26 and illustrate the ability of the pad 440 to elastically stretch due to the slits 446 in the solid or fused sections opening or expanding between fluid channels as the pad is stressed.

FIG. 27 is another embodiment of a heating/cooling pad 460 including thermal fluid channels 462 in a generally zigzag pattern and having a central slit 464 for flexibility and flow path control purposes, as well as individual slits 466 in fused or solid sections 468 of the pad 460 to provide multi-directional elasticity or stretching. A fluid input 470 and a fluid output 472 are provided as generally previously described. The stretching provided for by this embodiment is similar to that provided by the embodiment of FIG. 26 and allows lengthwise, widthwise and bi-directional diagonal stretching, as well as elasticity in other radial directions relative to a center of the pad 460.

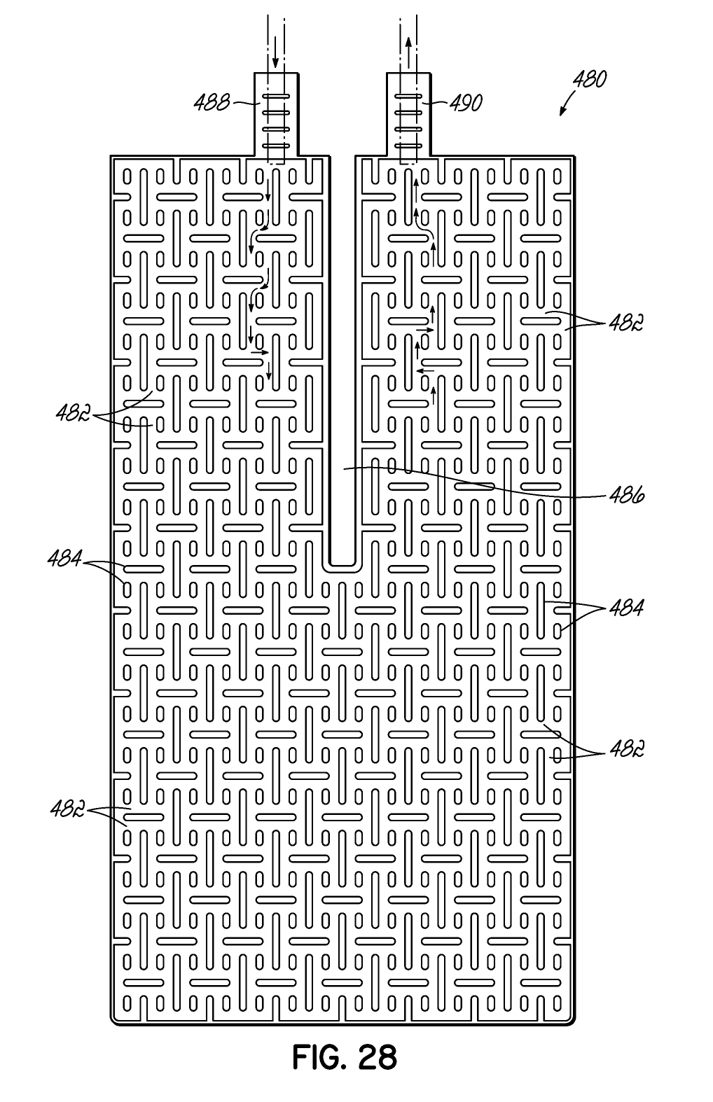

FIG. 28 illustrates another embodiment of a heating/cooling pad 480 with flow channels 482 provided between solid or fused sections 484 and a central slit 486, a fluid input 488 and a fluid output 490.

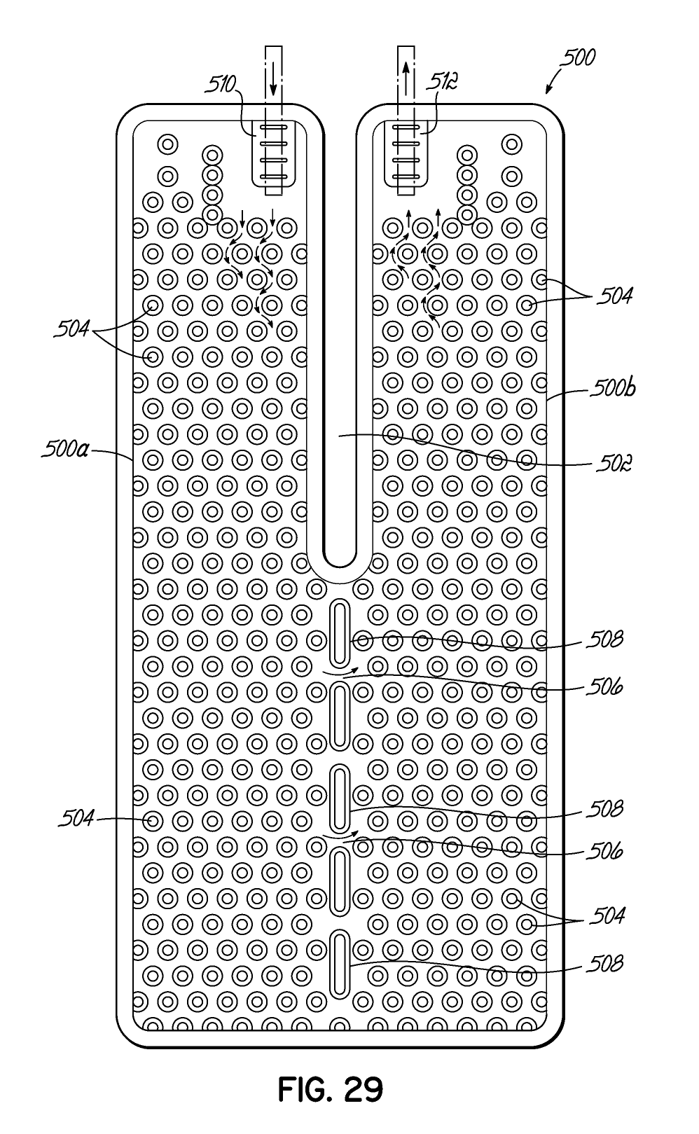

FIG. 29 illustrates another alternative heating/cooling pad 500 including similar left and right pad sections 500a, 500b as previously described separated by a central slit 502, and circular fused or solid sections 504 defining fluid paths therebetween. In addition, in a lower portion of the pad 500, as viewed in FIG. 29, additional fluid channels 506 are provided by fused or solid sections 508 of the pad 500 to provide multiple fluid paths transferring the fluid from the left section or half of the pad 500 to the right section or half of the pad 500. A fluid input 510 and a fluid output 512 are provided.

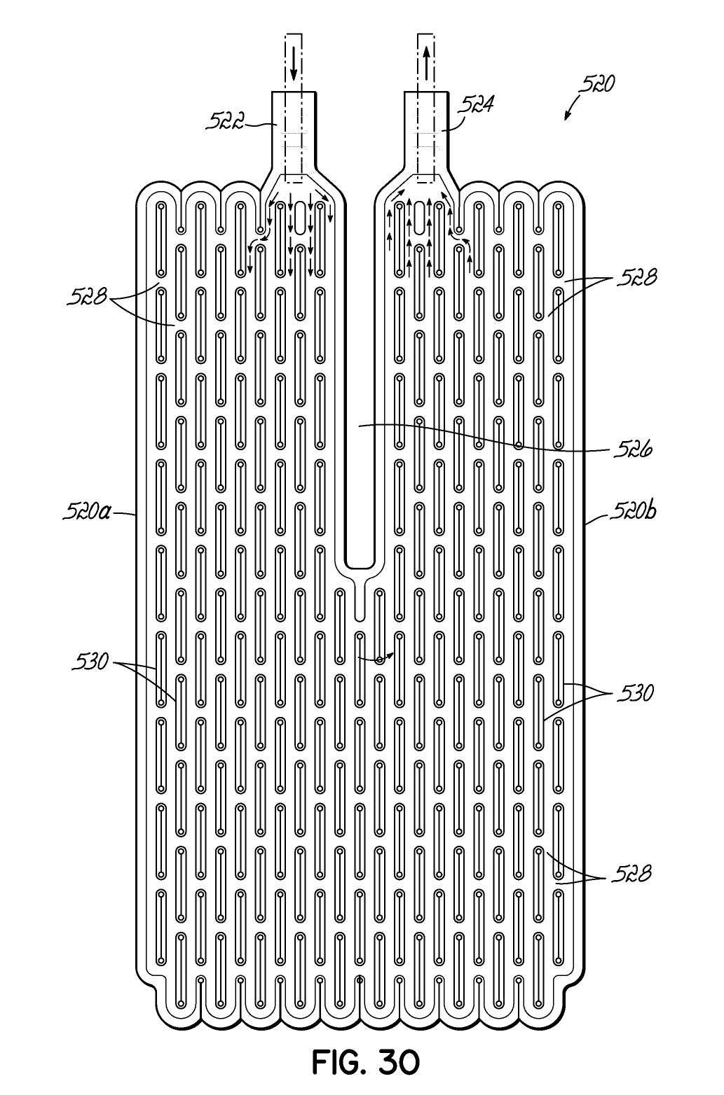

FIG. 30 illustrates another thermal pad 520 including a fluid input 522, a fluid output 524 and a central slot 526 between two pad sections or halves 520a, 520b, and fluid channels 528 formed between vertically oriented (lengthwise oriented) fused or solid sections 530.

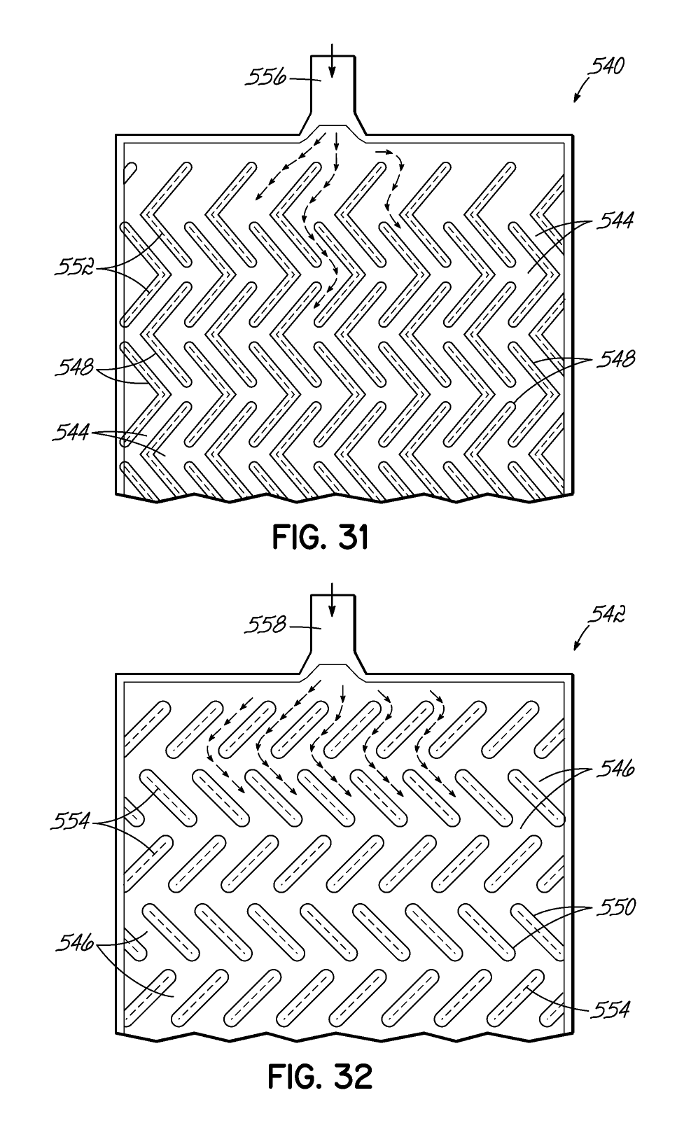

FIGS. 31 and 32 illustrate additional embodiments of the thermal pads 540, 542 having generally zigzag configurations of fluid channels 544, 546, separated by solid or fused sections 548, 550 of the pad and slits 552, 554 provided in the respective solid or fused sections 548, 550 to provide multi-directional elasticity, generally as previously described. Fluid inputs 556, 558 are shown but fluid outputs are not shown, but would be provided elsewhere in the pads 540, 542.

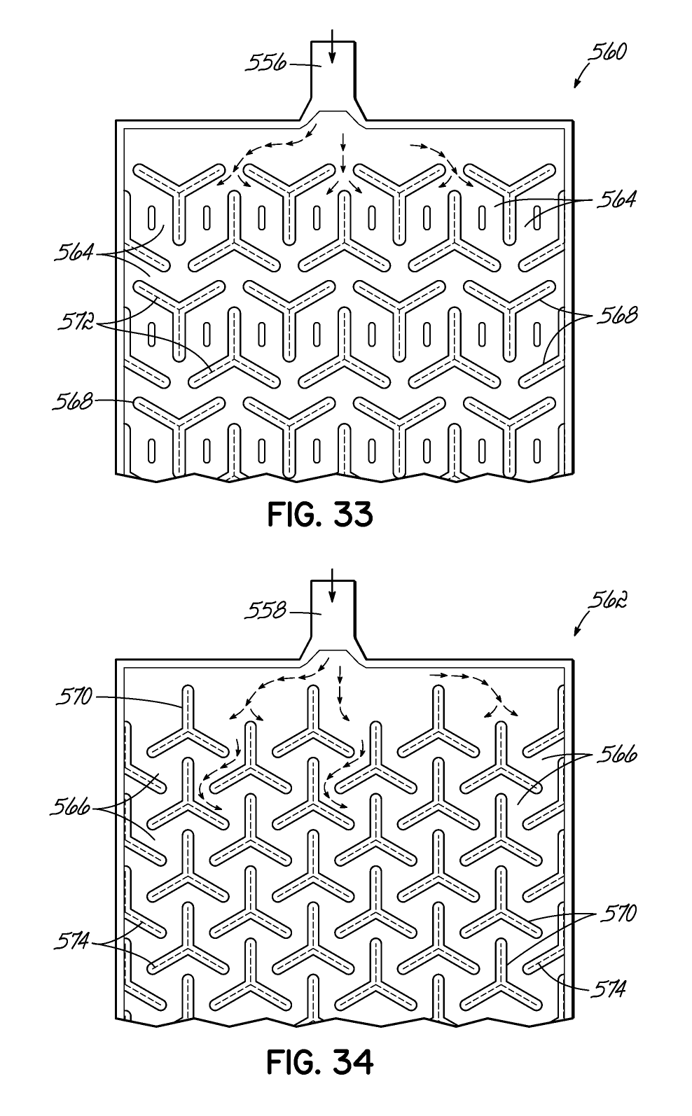

FIGS. 33 and 34 illustrate two additional alternative thermal pads 560, 562 embodiments exhibiting similar designs. In these designs, fluid channels 564, 566 are formed between multiple fused or solid sections 568, 570 of the pads 560, 562 configured into generally “Y” shapes. The Y-shaped fused or solid sections 568, 570 include slits 572, 574 in each segment of the “Y” for providing elasticity and flexibility in multiple directions as previously described.

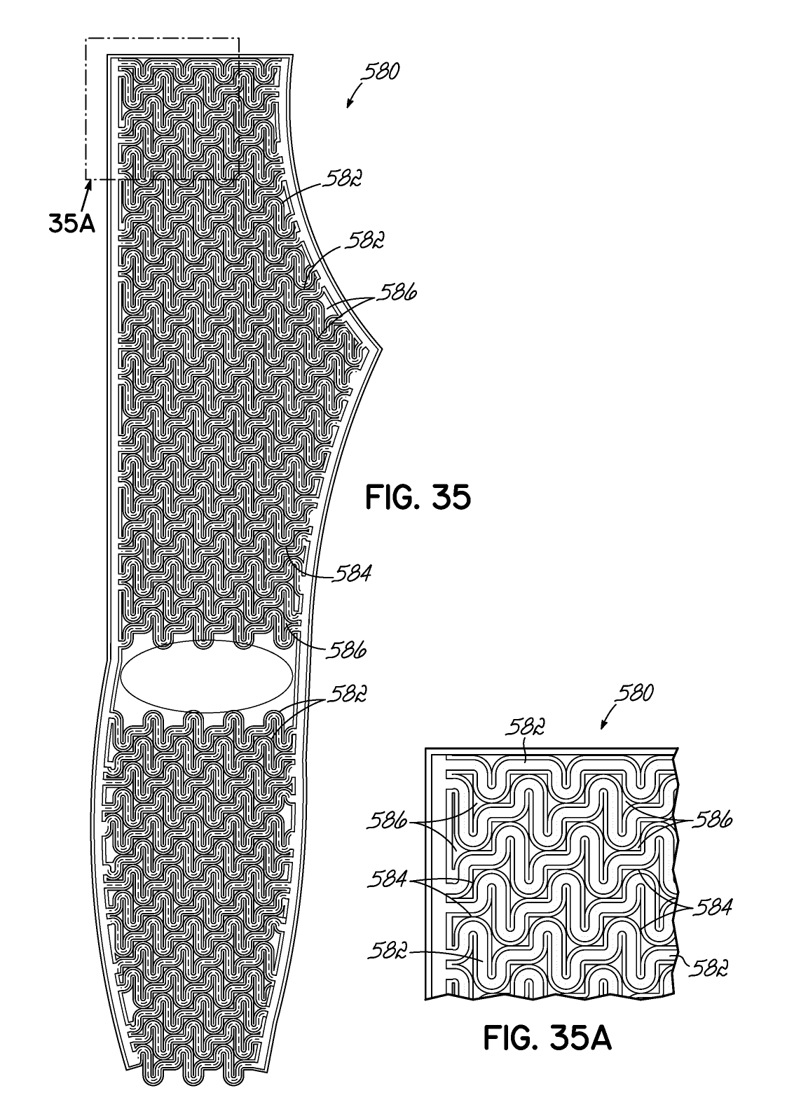

FIGS. 35 and 35A illustrate another alternative thermal pad 580 having a more complicated fluid passage design. These fluid passages are channels or tubes 582 that are at least sporadically disconnected from one another such that they may be stretched and spaced from one another to accommodate the size and shape of the individual user. Thus the exterior walls of the channels 582 may be connected to one another in certain locations 584, but disconnected at other locations 586 to allow the channels 582 to stretch and move relative to each other to accommodate the user.

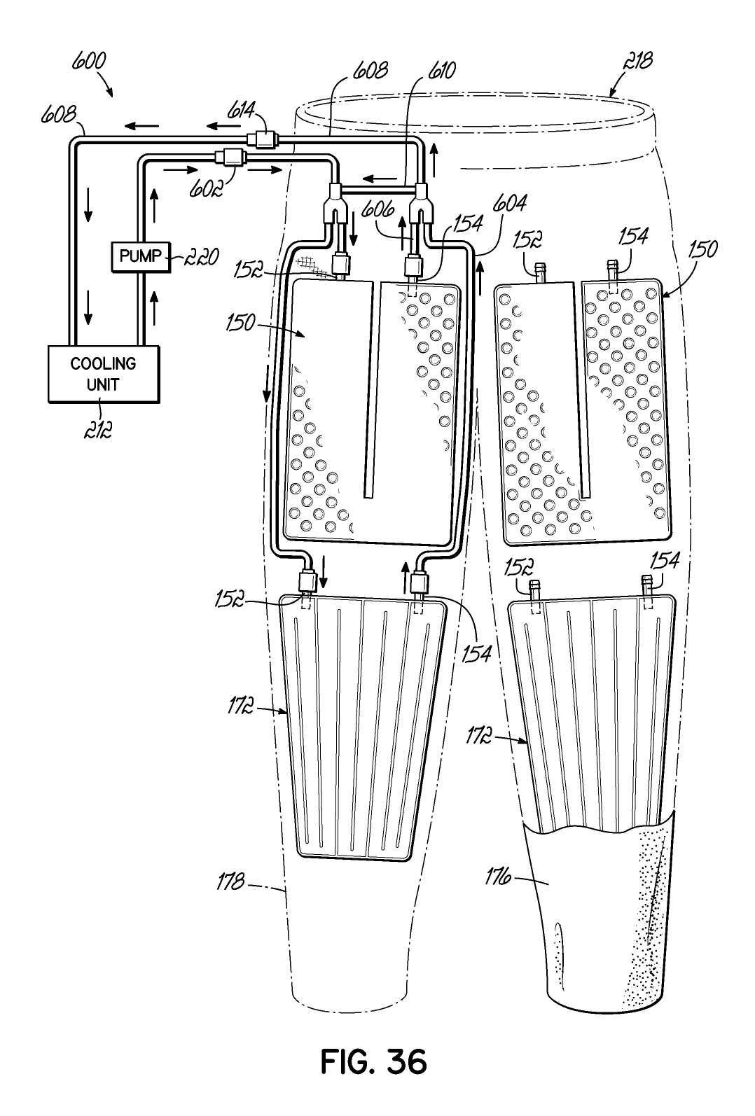

FIG. 36 illustrates another alternative system 600 showing a recirculation path provided by the tubing or conduit configuration. As shown, cold fluid, in this example, will be pumped from the cooling unit 212 by a pump 220. The cooling unit 212 may be a cold fluid reservoir (or another type of cooling unit). The thermal fluid is directed through an input fluid coupling 602 and into the particular cooling pads 150, 172 associated with the pressure garment 218. The cooling fluid will be returned through the conduits 604, 606, 608 as shown by the arrows and at least some of the fluid will then travel through a recirculation path provided by a conduit 610, which may or may not include a flow control device (not shown). The flow control device may include a shunt device that is not adjustable but provides for a certain amount of recirculated fluid to flow back into the input side for purposes of temperature control, or it may be adjusted as disclosed herein. The remainder of the fluid travels through the output conduit 608 and output fluid coupling 614 into the cooling unit 212. It will be appreciated that any of the other features described herein may be incorporated into this system 600 or, stated another way, this recirculation feature may be incorporated into any of the other system designs contemplated herein. As with the other recirculation systems described, this will provide for a certain amount of temperature elevation to the fluid being introduced into the pad/compression garment 218.

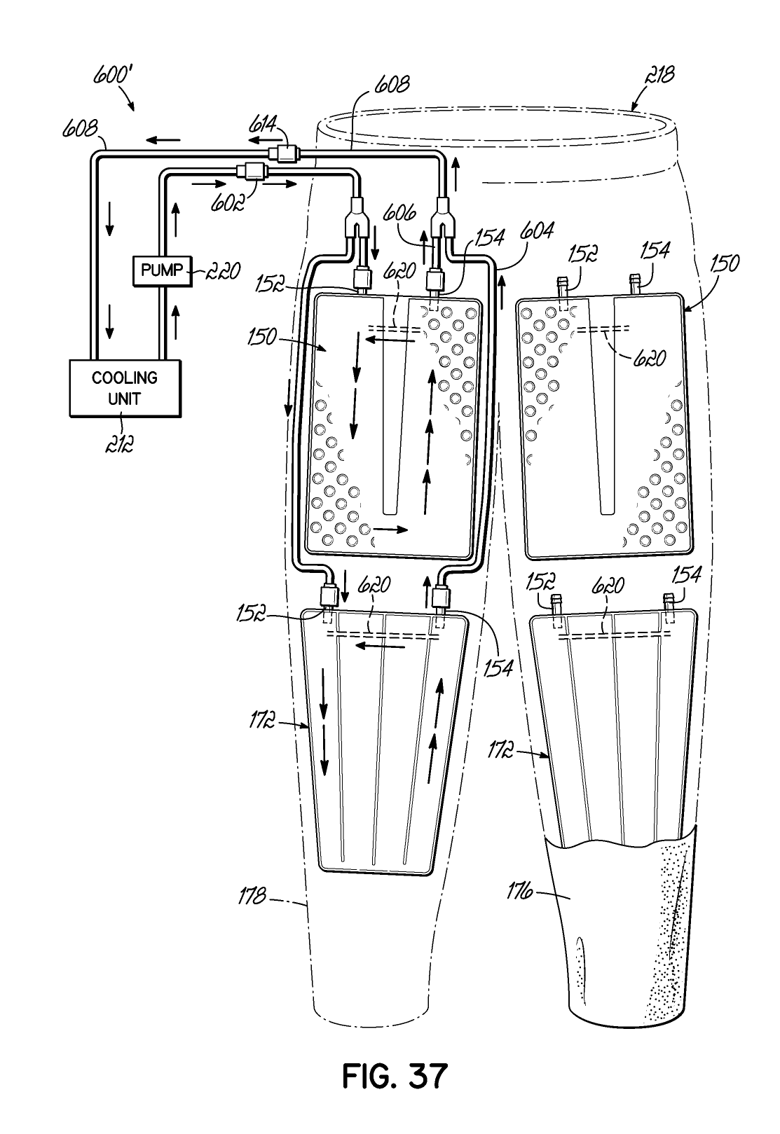

FIG. 37 illustrates a system 600′ similar to FIG. 36, but illustrating that the recirculation passage 620 (with or without a flow control device) may be incorporated directly into the cooling pads 150, 172, as shown by the dashed lines in the respective cooling pads 150, 172.

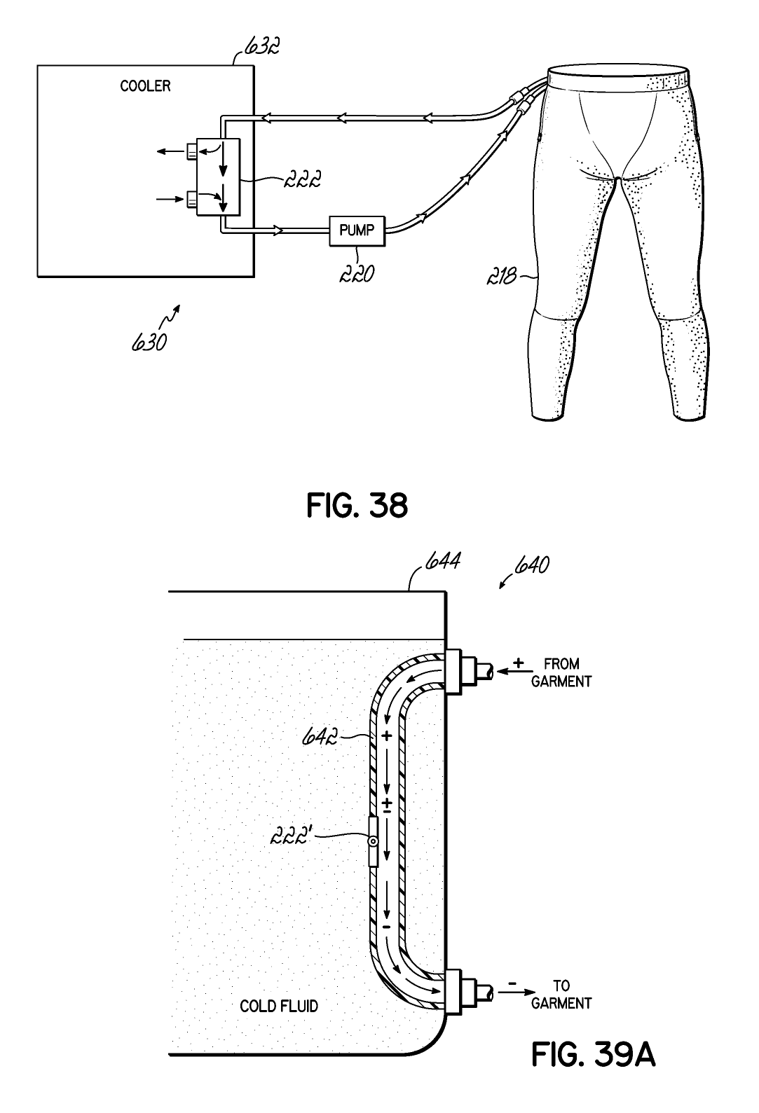

FIG. 38 illustrates another alternative system 630 showing a flow control device 222 directly in the cooler 632, or other cooling unit of the system, such as described herein. The flow control device 222 may be a static device, such as a static shunt, or may include an adjustable valve to change the amount of fluid recirculation and therefore allow for fluid temperature control as previously described.

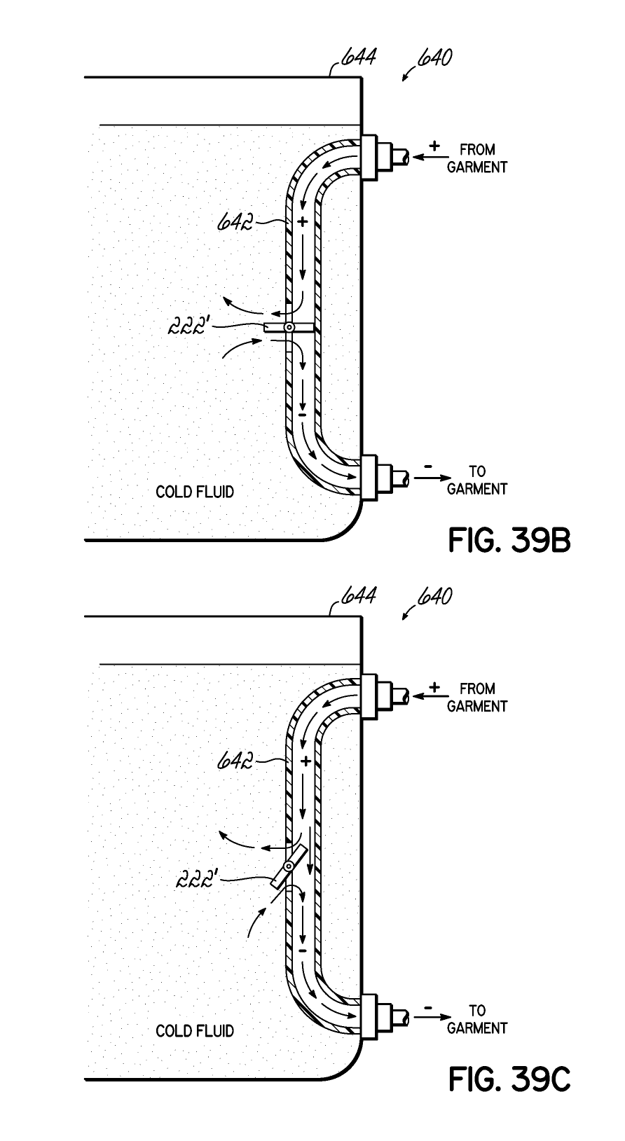

FIGS. 39A, 39B and 39C schematically illustrate another flow control device 222′ in the form of a valve structure for adjustably controlling the amount of fluid recirculating from the compression garment back into the input side of the cooling system 640. In FIG. 39A the fluid from the garment is being completely recirculated back into the garment through a recirculation passage or conduit 642, and no additional cold fluid is being introduced into the compression garment. In FIG. 39B, the valve 222′ is adjusted into a position that prevents any fluid from the garment from being recirculated back into the garment or input side of the system. FIG. 39C illustrates the valve 222′ adjusted to a position allowing some fluid to be recirculated back into the input side and other fluid to be directed into the cold fluid reservoir 644 for temperature control purposes.

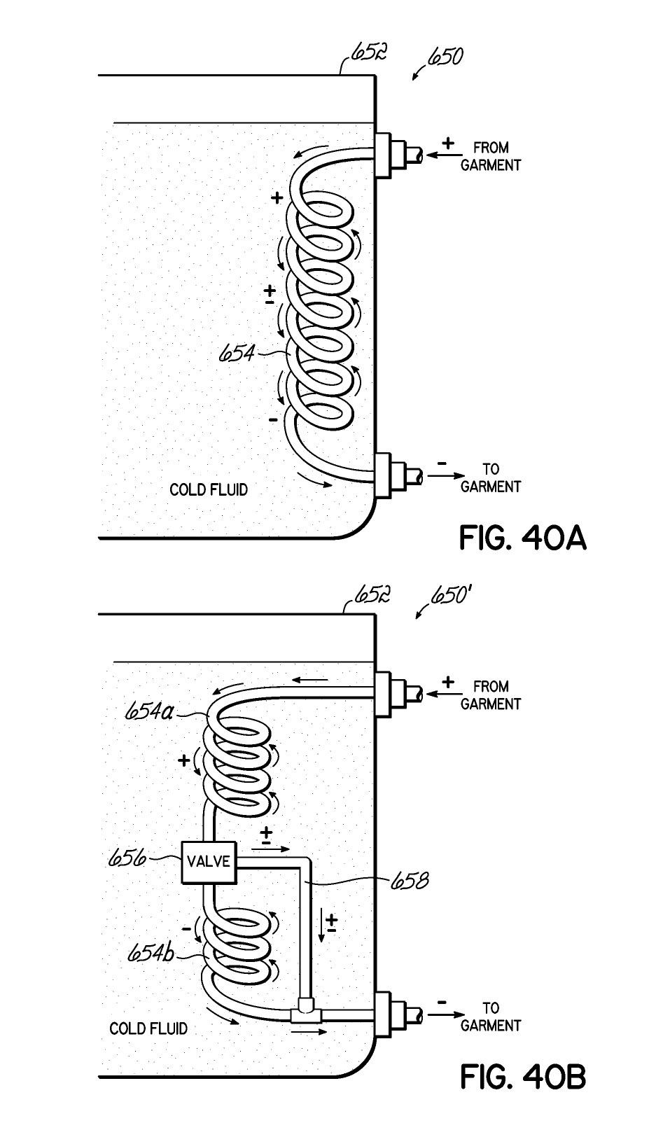

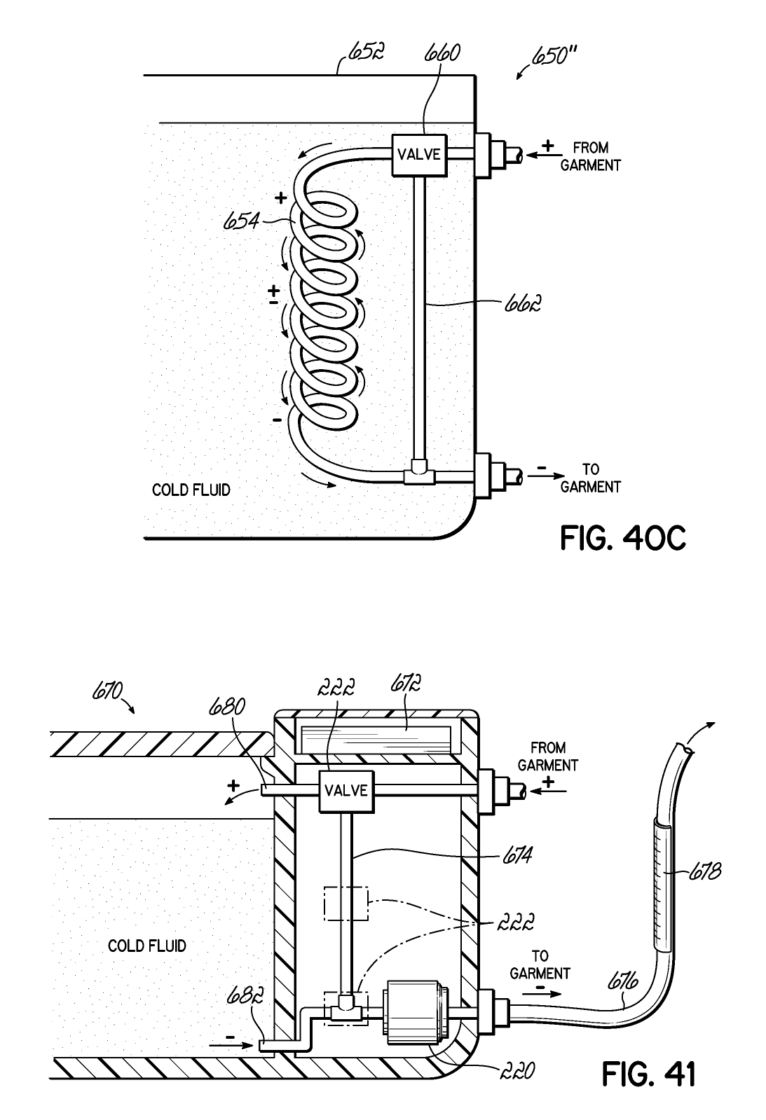

FIG. 40A schematically illustrates a system 650 with a cold fluid reservoir 652 including a coiled shunt 654 in the reservoir 652 that allows the cold fluid in the reservoir 652 to cool the recirculating fluid in the shunt 654 before that fluid is again delivered into the compression garment.

FIG. 40B illustrates an alternative system 650′ to the system 650 shown in FIG. 40A and further includes a valve 656 in the recirculation path. Specifically, the valve 656 controls the amount of recirculated fluid that is directed from a first coiled shunt portion 654a to a second coiled portion 654b to control the amount of cooling applied to the fluid. The valve 656 may be adjusted to bypass the second coiled portion 654b by the desired amount, through a bypass conduit 658, to adjust the amount of cooling effect on the recirculated fluid.

FIG. 40C is another alternative system 650″ to those shown in FIGS. 40A and 40B. In this system 650″, a valve 660 may be used to bypass a single coiled shunt 654 through a bypass conduit 662 to control the amount of cooling applied to the recirculated fluid. The valve may be adjusted such that some, all or none of the fluid entering from the compression garment travels through the coiled shunt 654 for cooling purposes prior to exiting the cold fluid reservoir 652 and being directed into the compression garment once again.

FIG. 41 Illustrates a cooling unit 670 that includes various integrated components, including a power supply, logic controller, user interface and other control components schematically shown as a control center 672 in an upper portion of the unit 670, a flow control valve 222 allowing a selected amount of fluid returning from the compression garment to be recirculated through a recirculation conduit 674 and a pump 220, back in to the compression garment. As shown in dash-dot lines, the flow control device 222 may have various locations in the recirculation path. An input conduit 676 provides the supply of cold fluid into the compression garment through the pump 220 which is integrated into the unit 670. A temperature sensor 678 is included to measure the temperature of the fluid directed into the garment. This sensor 678 may be of any desired type, such as the type that changes color when subjected to different temperatures, available from thermometersite.com (16 Level Thermometer). The sensor 678 may be used to determine how to adjust the flow control device 222 and achieve the desired fluid temperature. It will be appreciated that an automated temperature control system may be used with a suitable electronic temperature sensor (not shown) and control for the flow control device 222, heater(s) (not shown), pump 220 and other components of the unit 670 or other systems described herein. The cold fluid return line 680 may be located at any height above or below the cold fluid level, and the cold fluid input line 682 may be located at any height below the cold fluid level.

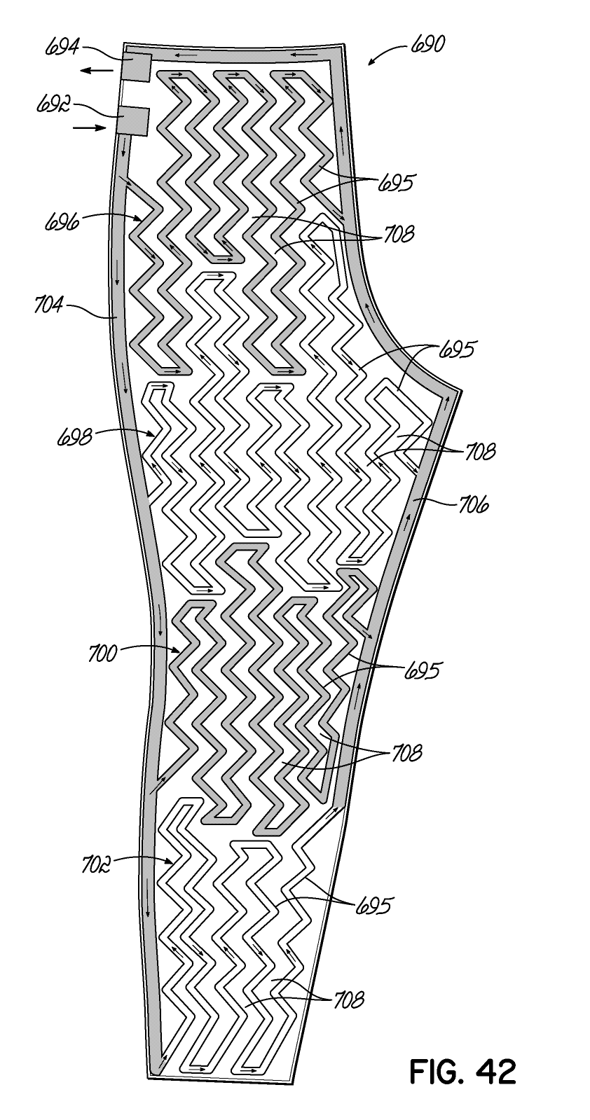

FIGS. 42, 43 and 44 illustrate elevational, schematic views of additional alternative embodiments for thermal pads. As with the other embodiments disclosed herein, the pads may be formed in any desired or necessary shape and configuration. These pads are illustrated as lower body pads, for example, for covering generally from the waist of the user to the ankle along the front or rear of one leg. The pad 690 illustrated in FIG. 42 includes an input 692 and an output 694 for the thermal fluid and individual tubular channels 695 formed generally in vertically oriented patterns and contained in separate sections 696, 698, 700, 702 of the pad 690. The fluid will flow through the input 692 down a main supply channel or conduit 704 and separately enter each section 696, 698, 700, 702 of the fluid channels 695 to traverse the pad 690 from left to right as shown in FIG. 42. The thermal fluid will exit each of the fluid channels 695 into a main output channel 706 in fluid communication with the output or outlet 692. As with other embodiments, these fluid channels 695 will allow multi-directional expansion and contraction of the pad 690 to conform to the individual user and allow better mobility. Thus, due to the zigzag or convoluted nature of the fluid channels 695, and the spaces 708 therebetween, these fluid channels 695 will allow the pad 690 to stretch and contract in lengthwise, widthwise and other generally radial directions relative to central locations of the pad 690.

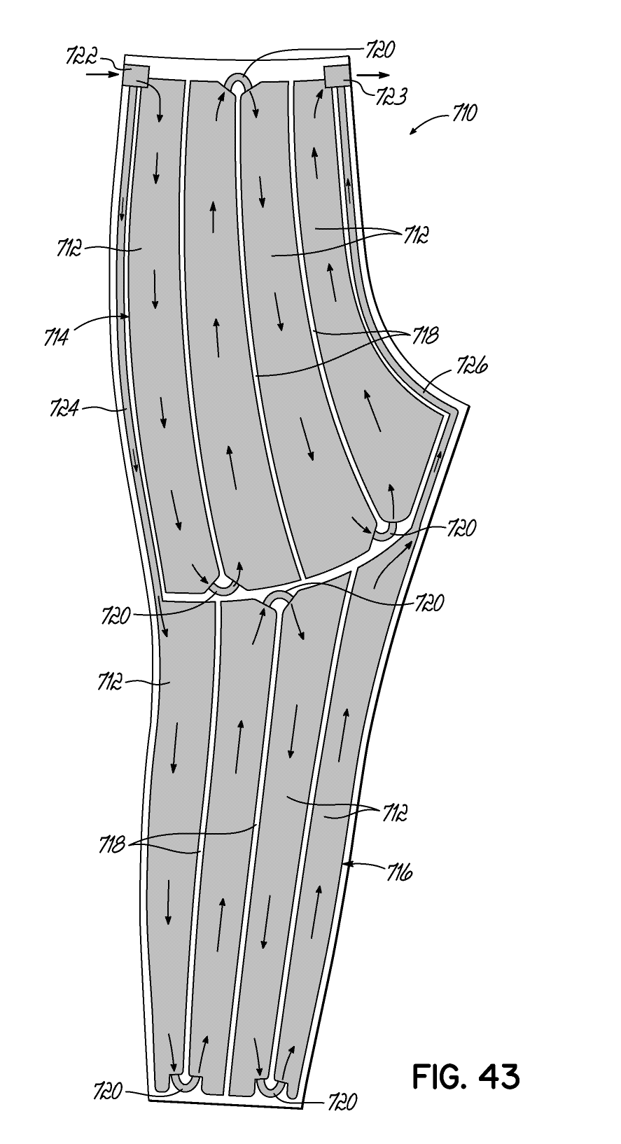

FIG. 43 illustrates a similar pad 710 to FIG. 42 in overall shape, but having a different configuration and construction of fluid channels 712. Generally, two fluid channel sections 714, 716 are shown and include respective fluid channels 712 in generally vertically oriented configurations. Gaps or elongate openings 718 are provided between adjacent fluid channels 712 and each fluid channel 712 is in fluid communication with an adjacent fluid channel 712 by way of connecting conduits 720. A fluid input or inlet 722 is provided as well as a fluid output or outlet 723. The thermal fluid flows into a first fluid channel 712, as well as into a main supply channel 724 to reach the first fluid channel 712 of the second section 716 of fluid channels 712. As mentioned, the fluid traverses the adjacent fluid channels 712 through the conduits 720 and exits either directly through the outlet or output 723 or through a main outlet conduit 726 and then through the outlet 723. The gaps or elongate openings 718 between the fluid channels 712 allow for multi-directional, elastic flexing, as do the connecting conduits 720.

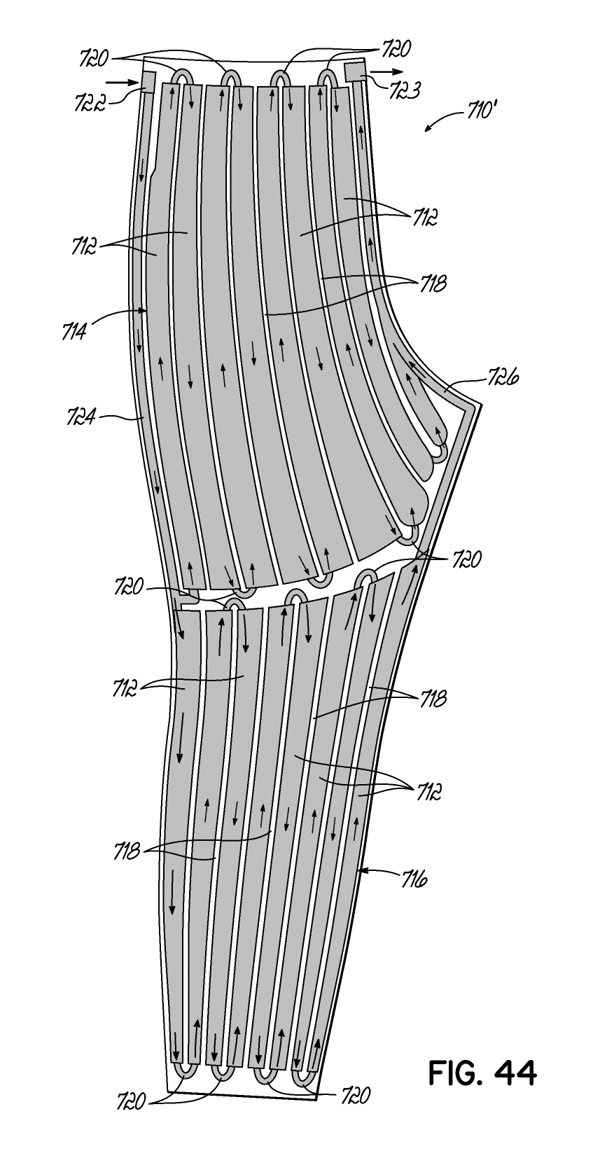

FIG. 44 illustrates a pad 710′ that is very similar to the pad 710 illustrated in FIG. 43 and, therefore, like reference numerals are used. Additional flexibility is obtained by the use of additional fluid channels 712, of narrower construction. In this manner the pad design includes a greater number of elongate openings or gaps 718 between the fluid channels 712 for providing the greater flexibility.

### Additional Treatments and Uses

Various treatments and uses of hot and cold therapy may be beneficial in many situations. As set forth below, some therapies and uses may benefit from use of the systems, devices and methods described above, while others may use other known heating and cooling systems, devices and methods. For example, a garment as generally disclosed herein may have separate heating and cooling regions for therapeutic purposes or other uses, such as those disclosed herein. One region of an upper garment, such as a shirt/jacket as shown in FIG. 1 may have a heater or hot fluid channels while a separate region of the garment may have cooling fluid channels. The first region may be one or both armpit regions of the garment, or wrist regions of the garment, and the second region may be the torso. The various regions may be cycled through hot and cold applications based on the needs or desired therapeutic effect, such as further described below. The pumps for pumping the thermal fluids may be cycled on and off to pulse the fluids and expand/contract the thermal fluid channels thereby creating a massaging or otherwise therapeutic effect. The thermal pad or pads in one or more section of the garment may be blocked off (such as by applying clips to block off the appropriate tubing/channels), so that localized treatment may be given for purposes such as treating an injury, for example. In this way, the same garment may have several different uses. Various additional treatments and uses are described below.

### Cooling with Shiver Control

Currently, therapeutic cooling is proven to save lives in MI and shock patients, but often requires the patient to be anesthetized and put on a ventilator. The present invention, in its various embodiments provides for cooling with no need for an anesthetic. Avoiding the anesthesia and obtaining 2 or 3 degrees C. of core cooling will allow the use of cooling in a less expensive and safer manner.

Cooling of the face and extremities rarely leads to shivering. In a bathtub or outside in cold air with an exposed torso, a person is more apt to shiver. Therefore, limiting and selecting the sites of cooling avoids the need for sedation/intubation and complication. Overall core cooling may be obtained with selective warming in shiver trigger areas. This could be accomplished with separate hot and cold circulation pads or a single pad with regions that receive hot and cold fluid. Alternatively a fluid cooling system could be used to cool and heating pads to warm separate regions of a person. This eliminates shivering while still providing the desired or necessary cooling of body portions. Also, compression of a region which is cool may limit the shiver response. The compression may be constant or intermittent to achieve this result. Also, slight skin stimulation with an electrical stimulus may prevent a shivering response. It may be possible to stimulate muscles to contract in a comfortable way. In this manner, the body may “believe” that it is shivering.

For myocardial ischemia, brain injury, and shock patients—regions of the patient could be cooled and warmed at the same time in different areas of the body. The systems and methods disclosed herein may be used to bring core temperature of the patient down. Shivering appears to occur less with limited or selective core warmth. Therefore, for example, selective heat may be applied under the arms, and/or in the neck area. Other areas of the body, such as extremities, face, wrists, etc., are cooled. For patients of this type, typically a compression suit or garment is difficult to use. Instead, easier delivery devices such as pads and wraps are used and the patient will lie on the bed. The pad can be placed under the patient, wrapped around the desired portion of the body, such as the neck, armpits, chest, abdomen, etc., for easier placement and use. Various types of fasteners may be used to hold the pad(s) in place, such as hook and loop, ties, snaps, etc.

Left/Right—the present invention may be used to cool one side or portion of the body and warm another. Warming and cooling may be alternated between left and right and different locations on the same or different sides of the body with the intermittent warming designed to prevent a shiver response, but with the net result being a cooling of the patient for therapeutic purposes. Cooling and warming could be rotated or cycled through different parts of the body to produce a systemic effect on temperature with a minimum in discomfort to the patient.

Warming may be quite gentle. For example, it is not necessary to warm to body temperature in order to feel warm under the arms. A slight increase in temperature relative to the temperature of the cold therapy (e.g., cold thermal fluid), will be helpful.

Also, some areas of the body are not sensitive to shiver. For example, the wrist is a good place to cool as the radial and ulnar arteries are at this location, and can carry the cooling effect to the body core.

Similarly, cold therapy may be applied over the femoral artery while warming the region. In this manner, the blood in the femoral artery carries the cooling effect, but the groin area does not feel cold to the individual

If a body area is cooled slowly with simultaneous warming episodes or regional warming, one may then be able to stop or reduce the warming after acclimatization to a reduced temperature level.

If cooling is applied to certain body parts, while warming others, this may allow the warming to eventually be stopped, or at least be intermittently stopped while maintaining shiver prevention.

Sensors may be used on the individual/patient for muscle twitch/shiver. Electromyography (EMG) is very sensitive to shiver. Therefore, before visible or vigorous shivering occurs, EMG may be used as an early detection, and action to prevent shivering, as disclosed herein, may be taken. An algorithm may be used in a suitable control system to reduce cooling or increase cooling to the individual/patient depending on readings from the EMG. If shivering is detected, either visibly or through the use of an electronic device such as EMG, then selective warming or a reduction in cooling may be applied with devices disclosed herein. For example, selective application of warmth (heat) to the armpit(s) may stop the shivering response upon detection. Temperature sensors, HR, EEG or index of brain function/alertness may be used as well.

It is also possible to combine drugs (such as clonidine, meperidine, BUSIPERON) with a selective warming application as disclosed herein to prevent shiver.

### Weight Control—BMR Adjustment

Shivering causes can cause a five times increase in BMR (basal metabolic rate). However, it is very uncomfortable to continuously shiver. Controlled cooling could be used to effect or cause an increase in BMR in a more comfortable fashion.

Animals in hibernation burn brown fat to stay alive and in humans fat accumulation can be decreased by increasing metabolic rate. Cooling increases caloric consumption by increasing BMR. A cooling system/method may be used to cool all or part of the body in a controlled fashion to increase calorie consumption. An electronic control may be implemented for expected calorie consumption vs. size/weight of person and expected weight loss may be estimated in this manner. Adding moisture to a cooled or heated area may reduce or increase the sense of cold or warmth. For purposes of adjusting the cooling level to allow for only a mild shiver, EMG may be utilized as discussed above. Activity sensor(s) may be added to help ensure that the individual/patient is actively moving to assist with weight loss, and a core temperature sensor may be added for safety. As with above discussion, selective warming may be used to prevent too much shiver response. A goal would be to tailor the amount of cooling (and re-warming) to optimize the effect. An option would be to cool selectively in different parts of the body. Cooling could be alternated and rotated or cycled between different parts of the body to increase metabolic rate and avoid discomfort or shivering. Alarms may be added for safety so that body temperature does not go too low, and the cooling time is not too long. For the same reasons, an automatic shut-off may be provided to prevent undesired health effects. A particularly useful combination would involve a healthy diet designed to reduce weight, and a cooling regimen to stimulate the burning of brown fat. Cooling and infusion or consumption of amino acids and proteins is a particularly beneficial approach. Additionally, breathing of humidified air with or without a mask may assist with the effect. Heat may also be used to reduce appetite.

### Psychologic Benefits, Stress Control

Heating and cooling therapy can also produce relaxation. This can be accomplished in various manners by mixing hot and cold therapy cycles to different parts of the body. For example, wraps or other types of garments may be used on the head, such as on the temples, neck, scalp, etc. Also, massaging and pulsing of compression may assist with the relaxation effect. An air bladder, such as described herein may be used to pulse hot and/or cold thermal fluids on selected areas of an individual's body. The thermal fluid pumps used in the invention may be pulsed for a therapeutic or massaging effect, or for other reasons. Other stimuli may be used in conjunction with heating and/or cooling to the individual as described herein. For example, visual stimulation such as displaying pleasing scenery, pleasing sounds or music, or other physical stimuli may be used.

Similar therapies or methods may be used to increase alertness of an individual. For example, pilots or other professionals who need to be particularly alert may have some body parts cooled and others warmed for increasing alertness and producing a high level of function. One combination would be to warm the arms, warm the groin but cool the torso.

This type of heating/cooling therapy may also aid in sleep disorders. A patient who cannot sleep may relax more and, sleep better in this way.

A system, such as disclosed herein, may be automated such that an individual may program cyclic heating and cooling to produce a calm state or an alert state.

### Nocturnal Breathing Disorders and Sleep Apnea Control and Snoring

Breathing is dependent on temperature. To stimulate respiration in a patient, selective cooling and warming may improve their breathing. Sudden applications of cold therapy may stimulate breathing. Cooling of the face, neck and chest regions may be particularly useful. Stimulating the same location on the body will result in acclimatization and then diminished or no further response to the sudden application of cold therapy. Therefore, it will be helpful to rotate the application of cold therapy to different body locations to prevent this effect, and retain the ability to effectively stimulate respiration. A suitable sensor may be used to detect when the patient has instances of apnea/snoring and cold therapy/stimulation may be applied in response to the detected condition. If a positive response is not achieved, then the temperature may be lowered. Cooling in the head and neck region may tighten up the muscles that cause airway obstruction (such as oropharangeal muscles) and thereby reduce the occurrence of obstructive sleep apnea. Another option is to apply warmth and then a sudden application of cold therapy, or vice versa, to prevent acclimatization. A collar or other apparel positioned around the neck, and/or jaw and facial region could be fashioned with cooling and heating elements. This could be covered with soft materials so it is comfortable and to allow a person a comfortable sleep and still provide heating and cooling to stimulate breathing

### Congestive Heart Failure

Selective cooling and warming could reduce the load on the heart. For example, warming extremities may reduce the need for blood flow to extremities so more blood may flow to the kidneys.

### Renal Failure and Poor Kidney Function

Selective warming and cooling could improve renal blood flow.

### Wound Healing

Blood flow may be increased in an extremity by selective warming of the area. For example, a leg ulcer might be healed better with regional warming. Optionally, warming the entire leg may assist by increasing bulk blood flow to the leg.

A combination of heat therapy and suction is another option to improve wound healing.

### Military

Soldiers working in cold areas could have selective warming at low rates and avoid shivering. They may actually become slightly core hypothermic but a even a small amount of warming in key body parts like the armpits may be all that is necessary to provide improved performance and comfort.

All of above (and especially relaxation therapy) could be accompanied by lighting control—e.g., control of low light level. Also, compression and massage may also assist, and garments as well as control systems may be designed to compress in cycles, rhythms, etc. As another option, a person may sit on a massage chair with cooling and warming added in accordance with the disclosure.

### Automation Designs

The entire cooling/heating system can be completely automated and controlled through a series of sensors, switches, and logic.

Various sensors can be incorporated into the system at any point (in garment, control unit, supply lines, user's body) to control the therapy provided by the system. Flow sensors can be incorporated into the fluid system as well as a pressure sensor, which could be used in both fluid and compression therapies. Temperature sensors can be employed to monitor the fluid temperature (at any point in the fluid circuit). Additional temperature sensors could monitor the body temperature of the user. Regional and/or central body temperatures could be monitored (for example bladder, esophageal, blood, rectal, and axillary temperatures). Heart rate and respiratory sensors could also be placed on the user's body. Electromyography can be used to monitor the skeletal muscle activity of the user, especially in the shivering prevention applications. Also to monitor a patient's shivering, a piezoelectric sensor (like an accelerometer) could be placed on the user or in the garment to monitor motion. In the reservoir(s), fluid level sensors can be used to prevent leaks and also to warn users that the fluid level is too low.

These sensors report their outputs to a central logic board, which can control the settings of the entire system. The flow and temperature characteristics can be controlled by valves (mechanically, electrically (solenoid valves for example), etc). The logic center can also control the start and stop of treatments. A timing device (like a timing relay) can be incorporated to control the start/stop times and duration of cycles of treatment (hot/cold/purge, etc) as well as the overall treatment time. These relays can also allow for pulsatile flow. Sample timing cycles are listed below:

Cold (15 mins)

Hot (15 mins)

Contrast: Cold (5 mins), Hot (5 mins), Cold (5 mins), Hot (5 mins) [or any variation, extension, etc]

Pulsatile flow: Pump on (5 sec), Pump off (2 sec) [or any variation, extension, etc]

An automatic safety shut off can be built into the logic so that the system stops at a certain time (20 minutes for example) to prevent misuse. A temperature sensor could also be employed to inhibit the use of the system if the user's body temperature is too high or low.

The logic board can also control the flow of hot or cold fluid to areas individually for shivering control. The sensors can be employed to adjust these settings according to the changing conditions of a patient's body. For example, if a motion sensor indicates a patient is shivering, the temperature in the axillary and neck (for example) regions is increased in response.

For the weight loss application, logic can be built in to predict the amount of weight lost based on the parameters of the system. For instance, a patient enters Body Mass Index, height, weight, etc., which allows system to predict the amount of weight lost. This could be reversed in a manner that allows the user to indicate how much weight they would like to lose, which would control the parameters of the system (i.e. temperature, amount of time on/off, etc).

A mechanical temperature switch (like a temperature sensitive valve) could direct flow based on the temperature of the fluid in the system.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A system for providing cooling or heating to one or more body parts of an individual, comprising a pair of compression pants and at least one pad coupled with the pants for heating and/or cooling the one or more body parts when contained in the pair of pants, the pad comprising:
a first flexible layer of material,
a second flexible layer of material secured to the first flexible layer of material, a plurality of spaced thermal fluid passages formed between the first and second flexible layers of material, the plurality of spaced thermal fluid passages provided in separate sections each including plural passages, an inlet in fluid communication with a main supply channel an outlet in fluid communication with a main outlet channel, whereby thermal fluid may be directed from the inlet separately into the plurality of thermal fluid passages of each of the sections and exit the outlet after travelling through the passages, and
a plurality of elongate openings in the pad, at least some of the elongate openings positioned in spaces between the thermal fluid passages, wherein the elongate openings allow the pad to be flexed in different directions to accommodate the one or more body parts of the individual, and wherein the at least one pad is held in contact with the one or more body parts in use by the pair of compression pants.

2. The system of claim 1 wherein the spaced thermal fluid passages and elongate openings are provided in a zigzag pattern.

3. The system of claim 1 wherein the at least one pad extends from a waist section of the pants to an ankle section of the pants along a leg section of the pants, the main supply channel and main outlet channel extending respectively along lengthwise edges of the pad.

4. The system of claim 1 further comprising a cooling unit for holding and cooling a thermal fluid, a pump fluidly coupled with the cooling unit to drive the thermal fluid in a fluid path from the cooling unit, through the inlet, main supply channel, thermal fluid passages, main outlet, and return channel, thereby circulating the cooled thermal fluid within the pad, and a recirculation passage fluidly coupled between the inlet and the outlet for directing an amount of the thermal fluid that has been circulated in the pad back into the inlet in order to raise the temperature of the cooled thermal fluid to a temperature above the temperature produced by the cooling unit.

5. The system of claim 4, further comprising:
a flow control device coupled in the recirculation passage for controlling the amount of thermal fluid directed back into the inlet.

6. The system of claim 5, wherein the flow control device further comprises an adjustable flow control device that is capable of selectively adjusting the amount of thermal fluid directed back into the inlet.

7. The system of claim 5, wherein the flow control device further comprises a non-adjustable flow control device that directs a predetermined amount of the thermal fluid back into the inlet.

8. The system of claim 5, further comprising a temperature sensor thermally coupled downstream of the recirculation passage for indicating the temperature of the thermal fluid entering the pad.
